SEQUENCE LISTING (2 sequences)

SEQ ID NO: 1 - protein, 317 aa, Homo sapiens
MRRASRDYTKYLRGSEEMGGGPGAPHEGPLHAPPPPAPHQPPAASRSMFVALLGLGLGQVVCSVALFFYFRAQMDPNRISEDGTHCIYRILRLHENADFQDTTLESQDTKLIPDSCRRIKQAFQGAVQKELQHIVGSQHIRAEKAMVDGSWLDLAKRSKLEAQPFAHLTINATDIPSGSHKVSLSSWYHDRGWAKISNMTFSNGKLIVNQDGFYYLYANICFRHHETSGDLATEYLQLMVYVTKTSIKIPSSHTLMKGGSTKYWSGNSEFHFYSINVGGFFKLRSGEEISIEVSNPSLLDPDQDATYFGAFKVRDID

SEQ ID NO: 2 - protein, 244 aa, Homo sapiens
MDPNRISEDGTHCIYRILRLHENADFQDTTLESQDTKLIPDSCRRIKQAFQGAVQKELQHIVGSQHIRAEKAMVDGSWLDLAKRSKLEAQPFAHLTINATDIPSGSHKVSLSSWYHDRGWAKISNMTFSNGKLIVNQDGFYYLYANICFRHHETSGDLATEYLQLMVYVTKTSIKIPSSHTLMKGGSTKYWSGNSEFHFYSINVGGFFKLRSGEEISIEVSNPSLLDPDQDATYFGAFKVRDID

(19) United States
(12) Patent Application Publication
(10) Pub. No.: US 2025/0025532 A1
(43) Pub. Date: Jan. 23, 2025

(54) METHODS OF BOOSTING THYMIC REGENERATION IN PATIENTS SUFFERING FROM A THYMIC INJURY BY USING RANKL

(71) Applicants: INSTITUT NATIONAL DE LA SANTE ET DE LA RECHERCHE MEDICALE, Paris (FR); UNIVERSITE D'AIX MARSEILLE, Marseille (FR); CENTRE NATIONAL DE LA RECHERCHE SCIENTIFIQUE (CNRS), Paris (FR)

(72) Inventors: Magali IRLA, Marseille - cedex 09 (FR); Noella LOPES, Marseille - cedex 09 (FR)

(21) Appl. No.: 18/762,774

(22) Filed: Jul. 3, 2024

Related U.S. Application Data
(63) Continuation-in-part of application No. 17/730,261, filed on Apr. 27, 2022.
Application No. 17/730,261 is a continuation of application No. 16/488,849, filed on Aug. 26, 2019, now Pat. No. 11,351,226.

(30) Foreign Application Priority Data
Feb. 27, 2017 (EP) 17305214.3

(51) Int. Cl. A61K 38/17; A61K 31/663; A61P 37/00

(52) U.S. Cl. CPC A61K 38/1793; A61K 31/663; A61P 37/00; C07K 2319/30

(57) ABSTRACT

Age-related thymic involution is a major cause of immunosenescence, characterized by reduced T-cell production, resulting in increased susceptibility to cancers, infections, autoimmunity and reduced vaccine efficacy. RANK/RANKL axis in the thymus is altered during aging and endothelial cells (EC) depend on RANK for their cellularity and functional maturation. Decreased RANKL availability during aging results in a decline in cellularity and function of EC and thymic epithelial cells (TEC) leading to thymic involution. Whereas RANKL neutralization in young mice mimics thymic involution, RANKL cytokine treatment in aged mice restores thymic architecture, EC and TEC cellularity and functional properties. Consequently, RANKL improves T-cell progenitor homing to the thymus and boosts T-cell production. This cascade of events results in peripheral T-cell renewal and effective anti-tumor and vaccine responses. RANKL stimulates EC and TEC in human thymic organo-cultures and is the basis of a treatment that rejuvenates thymic function and improves T-cell immunity in the elderly.

### SEQUENCE LISTING

This application includes as the Sequence Listing the complete contents of the accompanying XML file “11450582pa_sequence listing”, created Oct. 15, 2024, containing 3373 bytes, hereby incorporated by reference.

### FIELD OF THE INVENTION

The present invention relates to a method of boosting thymic regeneration in patients suffering from a thymic injury based on the administration of RANKL. The invention further relates to rejuvenating thymic function and improving T-cell mediated immune responses during aging.

### BACKGROUND OF THE INVENTION

The thymus controls the generation of T lymphocytes. Cortical thymic epithelial cells (cTECs) support the differentiation of T-cell progenitors and the conversion of CD4+CD8+double-positive (DP) thymocytes into CD4+CD8− and CD4−CD8+ single-positive (SP) cells (1). Medullary TECs (mTECs) purge the TCR repertoire of hazardous autoreactive T cells by expressing thousands of tissue-restricted antigens (TRAs), which are controlled by Aire (Autoimmune Regulator) and Fezf2 (Forebrain Expressed Zinc Finger 2) factors (2-5). Reciprocally, thymocytes sustain TEC differentiation and organization. This complex interplay is referred to as thymic crosstalk (6, 7).

Cytoablative treatments such as radiation or chemotherapy, used to prepare patients notably to bone marrow (BM) transplantation (BMT), severely affect not only hematopoietic cells but also TECs, which results in delayed T-cell reconstitution (8-11). Thymic injury triggered by total body irradiation (TBI) leads to profound alterations characterized by a drastic reduction in the cortex resulting from the massive depletion of DP thymocytes and a significant decrease in the medulla (12). Alterations in TEC ultrastructure and a reduction in some stromal cells have also been reported (10, 13).

The recovery of a fully competent T-cell compartment is therefore a prolonged process that is considerably delayed compared to that of myeloid, NK or B cells (14). This period of compromised immunity is prompt to serious clinical consequences such as opportunistic infections, autoimmunity or tumor relapse and could lead to post-transplant morbidity and mortality (8, 15-18). Identifying an effective treatment that acts at several levels by improving (1) the regeneration of TECs, which are critical for the renewal of stromal niches, (2) the recovery of cortical and medullary TECs, which control the different steps of thymopoiesis and (3) thymus homing of T-cell progenitors, which is important for T-cell recovery, is of paramount clinical interest to optimally boost thymic regeneration (19-21).

RANKL, a TNF family member, has emerged as an important regulator of epithelial cell growth and differentiation in different tissues such as mammary glands during pregnancy (22), the renewal and epidermal growth of the hair follicles (23) or M-cell differentiation from intestinal epithelial cells in Payer's patches (24). In the embryonic thymus, RANKL provided by lymphoid tissue inducer (LTi) cells and invariant Vγ5+TCR+ T-cell progenitors promotes the emergence of Aire+ mTECs, which mediate T-cell tolerance (4, 25, 26). In the postnatal thymus, RANKL produced by SP thymocytes enhances numbers of Aire+ mTECs and the size of the medulla (6, 27-31). However, although RANKL is a potent inducer of mTEC differentiation at steady state, whether and how RANKL drives thymic regeneration upon BMT remain unknown.

### SUMMARY OF THE INVENTION

The present invention relates to methods of boosting thymic regeneration in patients suffering from a thymic injury. In particular, the present invention is defined by the claims.

### DETAILED DESCRIPTION OF THE INVENTION

Cytoablative treatments lead to severe damages on thymic epithelial cells (TECs), which result in delayed de novo thymopoïesis and a prolonged period of T-cell immunodeficiency. Understanding the mechanisms that govern thymic regeneration is of paramount interest for the recovery of a functional immune system notably after bone marrow transplantation (BMT). Here, the inventors show that RANK ligand (RANKL) is strongly upregulated in LTi cells and to a lesser extent in CD4+ SP cells during the early phase of thymic regeneration. Importantly, whereas the administration of a neutralizing RANKL antibody severely alters TEC regeneration, the administration of RANKL protein after total body irradiation and BMT boosts the regeneration of cortical and medullary TEC subsets and thymic epithelial progenitor-enriched cells (TEPCs). RANKL treatment also enhances the thymus homing of lymphoid progenitors and de novo thymopoiesis. The inventors further found that RANKL administration increases specifically in LTi cells, lymphotoxin a (LTa), which is critical for both TEC regeneration and T-cell reconstitution after BMT. Notably, this treatment is also beneficial upon BMT in aged individuals. This study indicates that RANKL would be clinically useful to improve T-cell function recovery after BMT not only in young but also in aged individuals by controlling multiple facets of thymic regeneration.

Accordingly, the first object of the present invention relates to a method of boosting thymic regeneration in a patient suffering from a thymic injury comprising administering to the subject a therapeutically effective amount of a RANKL polypeptide.

As used herein, the term “thymic injury” has its general meaning in the art and refers to any insult of the thymic epithelial cells (TEC) and T cells. Typical insult includes cytoablative therapy (e.g. required to prepare patients to bone marrow transplantation (BMT)), complications related to HIV/AIDS, aging process, malnutrition, and radiation poisoning due to nuclear disaster. The term also includes age-related thymic involution, i.e. the progressive shrinking of the thymus with age.

The thymic injury may result from irradiation, for instance as medical treatment. Any subject/patient who receives irradiation may be treated with the method of the invention (i.e by administrating RANKL). Examples of diseases which are in part treated with irradiation included but are not limited to: cancers such as multiple myeloma, non-Hodgkin's lymphoma, Hodglin's disease, acute myeloid leukemia, neuroblastoma, ovarian cancer, germ-cell tumors, acute lymphoblastic leukemia, chronic myeloid leukemia, myelodysplasic syndromes, myeloproliferative disorders, chronic lymphocytic leukemia, juvenile chronic myeloid leukemia, and others diseases such as autoimmune disorders, amyloidosis, aplastic anemia, paroxysmal nocturnal hemoglobinuria, Fanconi's anemia, Blackfan-Diamond anemia, thalassemia major, sickle cell anemia, severe combined immunodeficiency, Wiskott-Aldrich syndrome, inborn errors of metabolism.

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at regular intervals, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

Typically, the term “patient” or “subject” is selected from the group consisting of children, young adults, middle aged adults, and the elderly adults. In some embodiments, the patient is an elderly patient, i.e. an adult patient sixty-five years of age or older. In particular, the administration of RANKL in elderly individuals is particular suitable for reversing the effects of aging on thymic involution. Indeed, the aging process results on an immune system which is less effective/functional which gives rise to cancers, infections, autoimmune diseases, etc. Typically, the patient (or “subject”) is a human.

In some embodiments, the patient suffers from cancer. As used herein, the term “cancer” has its general meaning in the art and includes, but is not limited to, solid tumors and blood borne tumors. The term cancer includes diseases of the skin, tissues, organs, bone, cartilage, blood and vessels. The term “cancer” further encompasses both primary and metastatic cancers. Examples of cancers that may treated by methods and compositions of the invention include, but are not limited to, cancer cells from the bladder, blood, bone, bone marrow, brain, breast, colon, esophagus, gastrointestinal, gut, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous; adenocarcinoma; mucocpidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; Sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malign melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioncuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In some embodiments, the patient has undergone a cytoablative therapy which caused thymic injury. The term “cytoablative therapy” has its general meaning in the art and refers to therapy that induces cytoablative effects on rapidly proliferating cells via several different mechanisms, ultimately leading to cell cycle arrest and/or cellular apoptosis. Typically, cytoablative therapy includes chemotherapy and radiotherapy.

As used herein, the term “radiotherapy” has its general meaning in the art and refers to the medical use of ionizing radiation, generally as part of cancer treatment to control or kill malignant cells.

As used herein the term “chemotherapy” has its general meaning in the art and refers to the medical use of chemotherapeutic agents effective in inhibiting tumor growth. Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaorarnide and trimethylolomelamine; acetogenins (especially bullatacin and bullatacinone); a carnptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cholophosphamide, estrarnustine, ifosfamide, mechlorethamine, mechlorethamine oxide hydrochloride, melphalan, novembichin, phenesterine, prednimus tine, trofosfamide, uracil mustard; nitrosureas such as carmustine, chlorozotocin, fotemustine, lomustine, nimustine, ranimustine; antibiotics such as the enediyne antibiotics (e.g. calicheamicin, especially calicheamicin (11 and calicheamicin 211, see, e.g., Agnew Chem Intl. Ed. Engl. 33: 183-186 (1994); dynemicin, including dynemicin A; an esperamicin; as well as neocarzino statin chromophore and related chromoprotein enediyne antiobiotic chromomophores), aclacinomysins, actinomycin, authramycin, azaserine, bleomycins, cactinomycin, carabicin, canninomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idanrbicin, marcellomycin, mitomycins, mycophenolic acid, nogalarnycin, olivomycins, peplomycin, potfiromycin, puromycin, quelamycin, rodorubicin, streptomgrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprinc, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophospharnide glycoside; aminolevulinic acid; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pento statin; phenamet; pirarubicin; podophyllinic acid; 2-ethylhydrazide; procarbazine; PSK®; razoxane; rhizoxin; sizofiran; spirogennanium; tenuazonic acid; triaziquone; 2,2′,2″-trichlorotriethylarnine; trichothecenes (especially T-2 toxin, verracurin A, roridinA and anguidine); urethan; vindesine; dacarbazine; mannomustine; mitobromtol; mitolactol; pipobroman; gacytosine; arabinoside (“Ara-C”); cyclophosphamide; thiotepa; taxoids, e.g. paclitaxel (TAXOL®, Bristol-Myers Squibb Oncology, Princeton, N.J.) and doxetaxel (TAXOTERE®, Rhone-Poulenc Rorer, Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylornithine (DMFO); retinoic acid; capecitabine; and phannaceutically acceptable salts, acids or derivatives of any of the above. Also included in this definition are antihormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and phannaceutically acceptable salts, acids or derivatives of any of the above.

In some embodiments, the administration of the RANKL polypeptide is performed after bone marrow transplantation. As used herein, the term “bone marrow transplantation” or “stem cell transplantation” used herein should be considered as interchangeable, referring to the transplantation of stem cells in some form to a recipient. The stem cells do not necessarily have to be derived from bone marrow but could also be derived from other sources such as umbilical cord blood. As used herein, the terms “hematopoietic stem cell transplantation” or “HSCT” refer to a component of the treatment of a wide array of hematologic disorders. Generally, there are two types of HSCTs: autologous and allogeneic transplantation. As used herein, the term “allogeneic” refers to deriving from, originating in, or being members of the same species, where the members are genetically related or genetically unrelated but genetically similar. An “allogeneic transplant” refers to transfer of cells or organs from a donor to a recipient, where the recipient is the same species as the donor. Allogeneic transplantation involves infusion of donor stem cells, typically using a donor that matches the recipient's MHC. As used herein, the term “autologous” refers to deriving from or originating in the same subject or patient. An “autologous transplant” refers to collection and retransplant of a subject's own cells or organs.

The method of the present invention is particularly suitable for preventing immunodeficiency linked to cytoablative conditioning. The method of the present invention is particularly suitable for the prophylactic treatment of infectious diseases, autoimmunity or cancer relapse in particular by boosting/enhancing the recovery of T-cell functions in a long-term manner.

The present invention also relates to a method for the prophylactic treatment of infectious diseases, autoimmunity or cancer relapse in particular by boosting/enhancing the recovery of T-cell functions in a long-term manner in a subject in need thereof comprising administering to the subject a therapeutically effective amount of a RANKL polypeptide. In other words, the invention also relates to a method for prophylactically treating cancer in a subject, comprising administering to the subject a therapeutically effective amount of a RANKL polypeptide, wherein the therapeutically effective amount is sufficient to stimulate recovery of antitumoral T-cell function.

As used herein, the term “prophylactic treatment” refers to any medical or public health procedure whose purpose is to prevent a disease. As used herein, the terms “prevent”, “prevention” and “preventing” refer to the reduction in the risk of acquiring or developing a given condition, or the reduction or inhibition of the recurrence or said condition in a subject who is not ill, but who has been or may be near a subject with the disease. It is also to be appreciated that the various modes of treatment or prevention of medical conditions as described are intended to mean “substantial,” which includes total but also less than total treatment or prevention, and wherein some biologically or medically relevant result is achieved.

As used herein the term “infectious disease” includes any infection caused by viruses, bacteria, protozoa, molds or fungi. In some embodiments, the infectious disease is an opportunistic infection. As used herein, the term “opportunistic infection” refers to bacterial, viral, fungal or protozoan infection caused by opportunistic pathogens that may or may not cause diseases in healthy hosts having a functioning immune system. These pathogens may cause an opportunistic infection since a compromised immune system presents an “opportunity” for such pathogens to thrive in an immuno-compromised subject. Non-limiting examples of viral infections include Herpes simplex virus (HSV) infections, Cytomegalovirus (CMV) infections, Varicella-zoster virus (VZV) infections, Human herpes virus 6 (HHV6) infections, Epstein-Barr virus (EBV) infections, respiratory virus infections (such as respiratory syncytial virus (RSV), parainfluenza virus, rhinovirus, and influenza virus) and adenovirus infections. Non-limiting examples of bacterial infections include Gram-negative bacteria infections such as Escherichia (e.g. Escherichia coli), Salmonella, Shigella, and other Enterobacteriaceae, Pseudomonas (e.g. Pseudomonas aeruginosa), Moraxella, Helicobacter, and Legionella infections. Non-limiting examples of fungal infections include Aspergillus infection (e.g. Aspergillus fumigatus), Candida infection (e.g. Candida albicans and non-albicans Candida) and other emerging fungi infections including Trichosporon, Alternaria, Fusarium, and Mucorales infections.

As used herein, the term “autoimmunity” is used to describe the process by which the body generates an immune response to self-antigens. Exemplary autoimmune diseases affecting humans include rheumatoid arthritis, juvenile oligoarthritis, collagen-induced arthritis, adjuvant-induced arthritis, Sjogren's syndrome, multiple sclerosis, experimental autoimmune encephalomyelitis, inflammatory bowel disease (for example, Crohn's disease and ulcerative colitis), autoimmune gastric atrophy, pemphigus vulgaris, psoriasis, vitiligo, type 1 diabetes, non-obese diabetes, myasthenia gravis, Grave's disease, Hashimoto's thyroiditis, sclerosing cholangitis, sclerosing sialadenitis, systemic lupus erythematosis, autoimmune thrombocytopenia purpura, Goodpasture's syndrome, Addison's disease, systemic sclerosis, polymyositis, dermatomyositis, acquired hemophilia, thrombotic thrombocytopenia purpura and the like.

As used herein, the term “RANKL” has its general meaning in the art and refers to Receptor Activator of Nuclear factor Kappa-B Ligand (RANKL) which is a protein that in humans is encoded by the TNFSF11 gene (Gene ID: 8600). The term RANKL is also known as tumor necrosis factor ligand superfamily member 11 (TNFSF11), TNF-related activation-induced cytokine (TRANCE), osteoprotegerin ligand (OPGL), and osteoclast differentiation factor (ODF), sOdf; CD254; OPTB2; TNLG6B; and hRANKL2. Exemplary amino acid sequences are represented by SEQ ID NO:1 (NCBI reference sequence NP_003692.1) and SEQ ID NO:2 (NCBI reference sequence NP_143026.1). RANKL is a compound that permits the regeneration of all TEC subtypes, i.e. cortical and medullary TECs as well as their progenitors, which are critical for the renewal of stromal niches. RANKL acts also on thymus homing of early thymic progenitors (ETPs) that give rise to the T-cell lineage. Thus, by acting on the availability size of stromal compartment and on the thymus homing of T-cell progenitors, RANKL importantly boosts de novo thymopoïesis.

| SEQ ID NO: 1 | |
|---|---|
| 1 | mrrasrdytk ylrgseemgg gpgaphegpl happppaphq ppaasrsmfv allglglgqv |
| 61 | vcsvalffyf raqmdpnris edgthciyri lrlhenadfq dttlesqdtk lipdscrrik |
| 121 | qafqgavqke lqhivgsqhi raekamvdgs wldlakrskl eaqpfahlti natdipsgsh |
| 181 | kvslsswyhd rgwakisnmt fsngklivnq dgfyylyani cfrhhetsgd lateylqlmv |
| 241 | yvtktsikip sshtlmkggs tkywsgnsef hfysinvggf fklrsgeeis ievsnpslld |
| 301 | pdqdatyfga fkvrdid |
| SEQ ID NO: 2 | |
| 1 | mdpnrisedg thciyrilrl henadfqdtt lesqdtklip dscrrikqaf qgavqkelqh |
| 61 | ivgsqhirae kamvdgswld lakrskleaq pfahltinat dipsgshkvs lsswyhdrgw |
| 121 | akisnmtfsn gklivnqdgf yylyanicfr hhetsgdlat eylqlmvyvt ktsikipssh |
| 181 | tlmkggstky wsgnsefhfy sinvggffkl rsgeeisiev snpslldpdq datyfgafkv |
| 241 | rdid |

As used herein, the term “RANKL polypeptide” refers to a polypeptide comprising an amino acid sequence having at least 80% of identity with SEQ ID NO:1 or SEQ ID NO:2. According to the invention a first amino acid sequence having at least 80% of identity with a second amino acid sequence means that the first sequence has 80; 81; 82; 83; 84; 85; 86; 87; 88; 89; 90; 91; 92; 93; 94; 95; 96; 97; 98; 99 or 100% of identity with the second amino acid sequence. Sequence identity is frequently measured in terms of percentage identity (or similarity or homology); the higher the percentage, the more similar are the two sequences. Methods of alignment of sequences for comparison are well known in the art. Various programs and alignment algorithms are described in: Smith and Waterman, Adv. Appl. Math., 2:482, 1981; Needleman and Wunsch, J. Mol. Biol., 48:443, 1970; Pearson and Lipman, Proc. Natl. Acad. Sci. U.S.A., 85:2444, 1988; Higgins and Sharp, Gene, 73:237-244, 1988; Higgins and Sharp, CABIOS, 5:151-153, 1989; Corpet et al. Nuc. Acids Res., 16:10881-10890, 1988; Huang et al., Comp. Appls Biosci., 8:155-165, 1992; and Pearson et al., Meth. Mol. Biol., 24:307-31, 1994). Altschul et al., Nat. Genet., 6:119-129, 1994, presents a detailed consideration of sequence alignment methods and homology calculations. By way of example, the alignment tools ALIGN (Myers and Miller, CABIOS 4:11-17, 1989) or LFASTA (Pearson and Lipman, 1988) may be used to perform sequence comparisons (Internet Program® 1996, W. R. Pearson and the University of Virginia, fasta20u63 version 2.0u63, release date December 1996). ALIGN compares entire sequences against one another, while LFASTA compares regions of local similarity. These alignment tools and their respective tutorials are available on the Internet at the NCSA Website, for instance. Alternatively, for comparisons of amino acid sequences of greater than about 30 amino acids, the Blast 2 sequences function can be employed using the default BLOSUM62 matrix set to default parameters, (gap existence cost of 11, and a per residue gap cost of 1). When aligning short peptides (fewer than around 30 amino acids), the alignment should be performed using the Blast 2 sequences function, employing the PAM30 matrix set to default parameters (open gap 9, extension gap 1 penalties). The BLAST sequence comparison system is available, for instance, from the NCBI web site; see also Altschul et al., J. Mol. Biol., 215:403-410, 1990; Gish. & States, Nature Genet., 3:266-272, 1993; Madden et al. Meth. Enzymol., 266:131-141, 1996; Altschul et al., Nucleic Acids Res., 25:3389-3402, 1997; and Zhang & Madden, Genome Res., 7:649-656, 1997.

In some embodiments, the RANKL polypeptide of the present invention is fused to an immunoglobulin domain so as to form an immunoadhesin. As used herein, the term “immunoadhesin” designates antibody-like molecules which combine the binding specificity of the RANKL polypeptide with the effector functions of immunoglobulin constant domains. In some embodiments, the immunoglobulin constant domain sequence in the immunoadhesin may be obtained from any immunoglobulin, such as IgG-1, IgG-2, IgG-3, or IgG-4 subtypes, IgA (including IgA-1 and IgA-2), IgE, IgD or IgM. In some embodiments, the immunoglobulin sequence is an immunoglobulin constant domain (Fc region). Immunoadhesins can possess many of the valuable chemical and biological properties of human antibodies. Since immunoadhesins can be constructed from a human protein sequence with a desired specificity linked to an appropriate human immunoglobulin hinge and constant domain (Fc) sequence, the binding specificity of interest can be achieved using entirely human components. Such immunoadhesins are minimally immunogenic to the patient and are safe for chronic or repeated use. The artisan skilled in the art can easily select the most appropriate Fc domain (Chan A C, Carter P J. Therapeutic antibodies for autoimmunity and inflammation. Nat Rev Immunol. 2010 May; 10(5):301-16. doi: 10.1038/nri2761. Review.). In some embodiments, the Fc region includes or not a mutation that inhibits complement fixation and/or Fc receptor binding (Zheng et al, Transplantation. 2006 Jan. 15; 81(1):109-16). In some embodiments, the Fc region is a native sequence Fc region. In some embodiments, the Fc region is a variant Fc region. In some embodiments, the Fc region is a functional Fc region. As used herein, the term “Fc region” is used to define a C-terminal region of an immunoglobulin heavy chain, including native sequence Fc regions and variant Fc regions. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. In some embodiments, the adhesion portion and the immunoglobulin sequence portion of the immunoadhesin are linked by a minimal linker.

According to the invention, the RANKL polypeptide is produced by conventional automated peptide synthesis methods or by recombinant expression. General principles for designing and making proteins are well known to those of skill in the art.

In some embodiments, it is contemplated that the RANKL polypeptide is modified in order to improve their therapeutic efficacy. Such modification of therapeutic compounds may be used to decrease toxicity, increase circulatory time, or modify biodistribution. For example, the toxicity of potentially important therapeutic compounds can be decreased significantly by combination with a variety of drug carrier vehicles that modify biodistribution. A strategy for improving drug viability is the utilization of water-soluble polymers. Various water-soluble polymers have been shown to modify biodistribution, improve the mode of cellular uptake, change the permeability through physiological barriers; and modify the rate of clearance from the body. To achieve either a targeting or sustained-release effect, water-soluble polymers have been synthesized that contain drug moieties as terminal groups, as part of the backbone, or as pendent groups on the polymer chain. For example, pegylation is a well-established and validated approach for the modification of a range of polypeptides. The benefits include among others: (a) markedly improved circulating half-lives in vivo due to either evasion of renal clearance as a result of the polymer increasing the apparent size of the molecule to above the glomerular filtration limit, and/or through evasion of cellular clearance mechanisms; (b) reduced antigenicity and immunogenicity of the molecule to which PEG is attached; (c) improved pharmacokinetics; (d) enhanced proteolytic resistance of the conjugated protein; and (e) improved thermal and mechanical stability of the PEGylated polypeptide.

By a “therapeutically effective amount” is meant a sufficient amount of RANKL polypeptide to provide therapeutic effects at a reasonable benefit/risk ratio applicable to any medical treatment. It will be understood that the total daily usage of the compounds and compositions of the present invention will be decided by the attending physician within the scope of sound medical judgment. The specific therapeutically effective dose level for any particular patient will depend upon a variety of factors including the disease being treated and the severity of the disease; activity of the specific compound employed; the specific composition employed, the age, body weight, general health, sex and diet of the patient; the time of administration, route of administration, and rate of excretion of the specific compound employed; the duration of the treatment; drugs used in combination or coincidental with the specific polypeptide employed; and like factors well known in the medical arts. For example, it is well known within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Preferably, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the patient to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

In some embodiments, the RANKL polypeptide is administered to the patient in combination with bisphosphonate to prevent bone resorption. As used herein, the term “bisphosphonate” means compounds characterized by two C—PO3 2-bonds. If the two bonds are located on the same carbon atom, the compounds are called geminal bisphosphonates. It should be noted that the term “bisphosphonate” as used herein is meant to also encompass diphosphonates, biphosphonic acids, and diphosphonic acids, as well as salts and derivatives of these materials.

Typically, the RANKL polypeptide is administered to the patient in the form of a pharmaceutical composition. The pharmaceutical composition comprises the RANKL polypeptide optionally with a pharmaceutically acceptable carrier or excipient. “Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. In the pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms. In some embodiments, the pharmaceutical composition is administered intrathymically. Preferably, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms. The RANKL polypeptide can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin. Sterile injectable solutions are prepared by incorporating the active polypeptides in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion. Some variation in dosage will necessarily occur depending on the condition of the subject being treated. The person responsible for administration will, in any event, determine the appropriate dose for the individual subject. The

RANKL polypeptide may be formulated within a therapeutic mixture to comprise about 0.0001 to 1.0 milligrams, or about 0.001 to 0.1 milligrams, or about 0.1 to 1.0 or even about 10 milligrams per dose or so. Multiple doses can also be administered. In addition to the compounds of the invention formulated for parenteral administration, such as intravenous or intramuscular injection, other pharmaceutically acceptable forms include, e.g. tablets or other solids for oral administration; liposomal formulations; time release capsules; and any other form currently used.

The invention will be further illustrated by the following figures and examples. However, these examples and figures should not be interpreted in any way as limiting the scope of the present invention.

### BRIEF DESCRIPTION OF THE FIGURES

FIG. 1. In vivo administration of RANKL substantially improves TEC regeneration after TBI. Flow cytometry profiles and numbers of total TECs (EpCAM⁺), cTECs (UEA-1⁻Ly51⁺), mTECs (UEA-1⁺Ly51⁻) and mTEC subsets (CD80^{lo}Aire⁻, CD80^{hi}Aire⁻ and CD80^{hi}Aire⁺) in UT WT mice or treated with PBS, GST or RANKL during three days upon SL-TBI.

FIG. 2A-F. RANKL boosts TEC regeneration and de novo thymopoïesis in an LTα-dependent manner upon BMT. (A) Experimental setup: WT CD45. 1:WT and WT CD45. 1:LTα^{-/-} chimeras were treated with GST or RANKL-GST proteins at d2, d4 and d6 after BMT and TEC regeneration and T-cell reconstitution were analyzed at d21 after BMT. (B) Expression level of LTα in thymic LTi cells (C) Thymic sections from WT CD45.1:WT and WT CD45.1:LTα^{-/-} mice treated with GST and RANKL at d2, d4 and d6 after BMT were stained for the expression of K14 at d21 pBMT. The histogram shows quantifications of medullary areas. m and c denote the medulla and the cortex, respectively. Twenty sections were quantified for each condition; Scale-bar: 100 μm. P-values were obtained by Student's t test. (D-E) Numbers of total TECs, cTECs and mTECs (D) and flow cytometry profiles of Aire⁺ mTECs (E). (F) Expression of mRNAs coding for TRAs (SP1 and SP2) in thymic stromal cells analyzed by qPCR.

FIG. 3A-E. Administration of risedronate in RANKL-treated mice prevents bone resorption without interfering with the beneficial effects of RANKL on thymic regeneration upon BMT. (A) TRAP staining of femur epiphysis and metaphysis analyzed at d21 pBMT from mice injected with GST, RANKL or RANKL+risedronate. 21, 12 and 24 sections were quantified for GST, RANKL and RANKL+risedronate conditions, respectively. Scale bar, 1 mm. B: bone; BM: bone marrow. (B-E) Numbers of total TECs, cTECs, mTECs (B), TEPC-enriched cells (C), total thymocytes and T-cell subsets (D) and ETPs (E) were analyzed at d21in the thymus of GST, RANKL and RANKL+Risodronate-treated mice. Data are pooled of 2independent experiments (n=3 mice per group). P-values were obtained by Student's t test.

FIG. 4A-D. Beneficial effects mediated by RANKL treatment on thymic regeneration upon BMT also require LTα expression in aged mice. (A-B) Numbers of total TECs, cTECs, mTECs (A) and TEPC-enriched cells (B) were analyzed at d21 upon BMT in the thymus from WT CD45.1:WT and WT CD45.1:LTα^{-/-} chimeras of 6-8 months of age treated with GST or RANKL proteins. (C-D) Numbers of total thymic cells, T-cell subsets (C) and ETPs (D) of CD45.1 origin. Data are pooled of 2 experiments (n=3 mice per group). P-values were obtained by Student's t test.

FIG. 5A-E. In vivo administration of RANKL shows benefits to reverse the effects of aging on thymic involution. (A) Representative pictures of thymus from WT mice of 12 months of age at d21 after administration of GST or RANKL proteins during three consecutive days. (B-E) Numbers of total TECs, cTECs, mTECs (B), TEPC-enriched cells (C), total thymic cells and T-cell subsets (DN, DP, CD4⁺ SP and CD8⁺ SP) (D) and ETPs (E) were analyzed at d21 in the thymus from untreated WT mice of 6 weeks of age or of 12 months of aged treated with GST or RANKL-GST during three consecutive days. Data are pooled of 4 independent experiments (n=3 mice per group). P-values were obtained by Student's t test.

FIG. 6. RANK is mainly expressed in the thymic medulla both in mouse and human. Mouse and human thymic sections were stained with anti-RANK (green) and anti-K14 (red) antibodies and counterstained with DAPI (blue). m and c denote the medulla and cortex, respectively. Scale bar, 5 mm.

FIG. 7A-G. Thymic rejuvenation in RANKL-treated aged mice. (A) Experimental procedure: aged (12-month-old) WT mice were injected i.v with GST (control) or RANKL proteins at day 0, 1, and 2 and their thymi analyzed at day 21. (B) Quantification of keratin-free zones. (C, D) Numbers of total thymic cells (E), cTEC (EpCAM⁺UEA-1⁻Ly51⁺), mTEC (EpCAM⁺UEA-1⁺Ly51⁻), thymic epithelial progenitor-enriched cells (TEPC; α6-integrin^{hi}Sca-1^{hi} in the TEC^{lo} subset) and Aire⁺ mTEC analyzed in CD45^{neg}-enriched cells (D). (E, F) Proportions among cTEC, mTEC^{lo} (CD80⁻ mTEC) (E), flow cytometry profiles and proportions among mTEC^{hi} (CD80⁺ mTEC) (F) of active-caspase-3⁺ cells. (G) Numbers of DN (CD4⁻CD8⁻), DP (CD4⁺CD8⁺), CD8SP (CD4⁻CD8⁺), CD4SP (CD4⁺CD8⁻), CD25⁺ TregP (CD4⁺CD8⁻CCR7⁺CD73⁻CD25⁺Foxp3⁻) and developing CD25⁺Foxp3⁺ Treg (CD4⁺CD8⁻CCR7⁺CD73⁻CD25⁺Foxp3⁺) cells. n=5-10 for 8-week-old, 5-16 for GST- and 5-16 for RANKL-treated aged mice in at least 3 independent experiments. Error bars show mean±SD, ns: not significant, *p<0.05, **p<0.01, ***p<0.001, ****p<0.0001 using Mann-Whitney test.

FIG. 8A-B. RANKL treatment restores EC functionality and T-cell progenitor homing to the thymus. (A) Experimental procedure of BM adoptive transfer experiments: WT CD45.2 aged mice were injected i.v with GST (control) or RANKL proteins at day 0, 1, and 2. Young and aged treated mice were injected i.v. with 35×10⁶ CD45.1 congenic BM cells at day 9 and their thymi were analyzed at day 10 for short-term homing assays of circulating progenitors. Alternatively, mice received congenic CD45.1/2 BM at day 5 and their thymi were analyzed four weeks later for long-term thymopoiesis assays. (B) Numbers of donor total cells and ETP (CD4⁻CD8⁻Lin⁻CD25⁻CD44⁺c-Kit⁺) in recipient thymi normalized to young mice in short-term homing assays.

FIG. 9. Flow cytometry profiles of donor CD4SP and CD8SP thymocytes in long-term thymopoiesis assays, normalized proportions and numbers of donor CD4SP and CD8SP cells among total thymocytes.

FIG. 10. Flow cytometry profiles, normalized proportions and numbers of donor splenic CD4⁺ and CD8⁺ T cells among total splenocytes in long-term assays in untreated young (8-week-old) and GST- or RANKL-aged (12-month-old) treated mice. n=6-11 for 8-week-old, 5-14 for aged-GST and 5-10 for aged-RANKL mice in at least 3 independent experiments. Error bars show mean±SD, ns: not significant, *p<0.05, **p<0.01, ***p<0.001,****p<0.0001 using Mann-Whitney test (I-K).

FIG. 11A-D. RANKL-induced thymic rejuvenation in aged mice improves T-cell responses in melanoma and immunization models. (A) Experimental procedure: 12-month-old WT mice were treated with GST or RANKL proteins at day-21, -20 and -19. Twenty-one days later, B16F10 melanoma cells were intradermally injected to these mice. Anti-PD1 and anti-CTLA-4 antibodies or their isotype controls were administrated at days 7,10,13 and 16. Tumor growth was assessed every two days from day 10. (B-D) Analysis of tumor growth (B), final tumor volume (C) and tumor volume classification (D) in GST and RANKL-treated aged mice.

FIG. 12. (A) Analysis of tumor growth in GST-treated aged mice receiving an anti-PD1 antibody, a combination of anti-PD1 and anti-CTLA-4 antibodies or their isotype controls and in RANKL-treated aged mice. (B) Densities (absolute numbers per mm³) of CD3⁺, CD8⁺CD3⁺, CD4⁺CD3⁺Foxp3⁻ (CD4⁺ Tconv) and CD4⁺CD3⁺Foxp3⁺ (Treg) TILs in tumors at day 21 in GST and RANKL-treated aged mice. (C) Normalized CD8⁺/Treg and CD4⁺ Tconv/Treg ratios in these mice.

FIG. 13. (A) Flow cytometry profiles and quantification of PD-1 expression levels in CD8⁺ TILs. (B) Flow cytometry profiles and densities of IFNγ-producing CD8⁺ TILs. (C) Densities of Gzmb-producing CD8⁺ T cells.

FIG. 14. (A) Flow cytometry profiles and densities of IFNγ-producing CD4⁺ TILs. (B) Pie-charts show the composition in tumor-infiltrating immune cells in both groups of mice. n=17 for aged-GST, 16 for aged-RANKL, 13 for aged-GST+anti-PD1 and 7 for aged-GST+anti-PD1+anti-CTLA-4 mice in 2 to 5 independent experiments (13B-C and 12A), 10-15 mice in 4 independent experiments (12B-C and 13A) and 4-7 mice per group in 2-3 independent experiments (13B-C and 14A).

FIG. 15. (A) Experimental procedure: aged (12-month-old) WT mice were treated with GST or RANKL proteins at day −28, −27 and −26. 28 days later, mice were immunized s.c. with OVA₃₂₃₋₃₃₉ peptide in CFA. (B) Flow cytometry profiles, proportions and numbers of OVA-tetramer⁺ CD4⁺ T cells. (C) Titers of anti-OVA IgG1 in the serum of these mice. Error bars show mean±SEM (B and E) or mean±SD (F-K, N,O). ns: not significant, *p<0.05,**p<0.01, ***p<0.001 using Mann-Whitney test or two-way ANOVA for tumor growth (B, E).

### EXAMPLES

The following Examples provide exemplary compositions and methods of the invention. The Examples also describe materials and methods for using embodiments illustrated in the figures. Additional details can be found in the section entitled “BRIEF DESCRIPTION OF THE FIGURES”.

### Example 1

### Material & Methods

### Human Sample

Human thymic fragments were obtained from normal male and female patients (3months to 15 years old) during cardiovascular surgery at the Necker Hospital (Paris, France). All tissue samples were fast-frozen in liquid nitrogen within 30 minutes of their excision from patients.

### Mice

CD45.1 and CD45.2 WT (Janvier), CD45.2 LTα^{-/-} (34), Rag2^{-/-} (Shinkai Y et al. Cell. 1992. PMID:1547487), ZAP-70^{-/-} (Kadlecek T A et al. J Immunol. 1998. PMID: 9794398) and Rorc^{GFP/GFP} knock-in (58) mice were on B6 background and maintained under specific pathogen-free conditions at the CIML (France). Chimeras were generated at 6-8 weeks or 6-8 months of age. WT mice of 12 months of age were used to analyze the effects of RANKL treatment on thymic involution.

### Thymic Damage and BM Chimeras

TBI was performed with a Cs-137 γ-radiation source. Sublethal-TBI (SL-TBI) was performed with 500 rads with no hematopoietic rescue and lethal-TBI (L-TBI) with 2 doses of 500 rads. For the generation of chimeras, 10⁷ BM cells of CD45.1 origin were injected i.v. into lethally irradiated (2×500 rads) CD45.2 WT or LTα^{-/-} recipient mice.

### RANKL and IL-22 Stimulations

The recombinant mouse RANKL-GST protein was produced as previously described (24). RANKL-GST (5 mg/kg) or GST (5 mg/kg) proteins were administrated i.v. daily during three days after SL-TBI or at day 2, 4 and 6 after BMT. Unmanipulated WT mice of 9-12 months of age were administrated i.v. with GST (5 mg/kg) or RANKL-GST (5 mg/kg) proteins during three consecutive days. Recombinant mouse IL-22 protein (200 μg/kg; R&D Systems) was administrated i.v. at days 2, 4, and 6 after BMT in combination or not with RANKL-GST protein.

### RANKL Neutralization Experiments

Endotoxin anf azide-Free (LEAF) neutralizing anti-RANKL antibody (7.5 mg/kg; IK22/5; BioLegend) or purified Rat IgG2a, κ isotype control (7.5 mg/kg; RTK2758; BioLegend) were administrated i.v. during three days after SL-TBI.

### Risedronate Treatment

WT mice were lethally irradiated, BM transplanted at day 0 and treated i.p. with risedronate (30 μg/kg; Sigma Aldrich) at day −2, −1, 0, 2, 4 and 6 before and after BMT. These mice were co-treated i.v. with GST or RANKL-GST proteins (5 mg/kg) at day 2, 4 and 6 after BMT.

### TRAP Staining

Mouse femurs fixed in 4% paraformaldehyde during 48 h were decalcified with 10% EDTA, pH 7.5 during two weeks. 5 μm sections were deparaffinized and stained for TRAP activity (Sigma Aldrich) according to the manufacturer's instructions. Sections were counterstained with hematoxylin and images were quantified with ImageJ software.

### TEC Isolation

TECs were isolated as previously described (29) by enzymatic digestion with collagenase D and DNase I (Roche) and depletion of hematopoietic cells using anti-CD45 magnetic beads and AutoMACS (Miltenyi Biotec).

### Flow Cytometry

CD4 (RM4.5), CD8α (53-6.7), CD45.1 (A20), LTα (AF.B3), IL-7Rα (SB/199), CD80(16-10A1), Ly51 (BP-1), I-Ab (AF6-120.1), CD45 (30-F11), CD44 (IM7) and Sca-1 (D7) antibodies were from BD. CD25 (PC61), RANK (R12-31), RANKL (IK22/5), CD3ε (145-2C11), lineage cocktail (145-2C11, RB6-8C5, M1/70, RA3-6B2, Ter-119), CD11c (N418), α6-integrin (GoH3) and CD31 (390) were from BioLegend. Foxp3 (FJK-16s), Ki-67 (SolA15), EpCAM (G8.8), LTBR (ebio3C8), Aire (5H12), RORγt (B2D), CD117 (2B8) and PDGFRα (APA5) were from eBioscience. FITC-conjugated UEA-1 was from Vector Laboratories. For RANKL and LTα detection, cells were incubated for 3 h with Brefeldin A (Biosciences). Foxp3 and Ki-67 intracellular stainings were performed with the Foxp3 staining kit (eBioscience). Aire, LTα, RANKL and RORγt intracellular stainings were performed with BD Cytofix/Cytoperm and Perm/Wash buffers. For staining with LTβR-Fc, cells were incubated with LTβR-Fc (RnD systems) at 1 μg/10⁶ cells for 45 min on ice. LTβR-Fc staining was visualized using a Alexa Fluor 488-conjugated donkey anti-human IgG F(ab′)₂ fragment (Jackson ImmunoResearch). Flow cytometry analysis was performed with a FACSCanto II (BD) and data were analyzed with FlowJo software.

### Quantitative RT-PCR

Total RNA was prepared with TRIzol (Invitrogen). cDNAs was synthesized with oligo(dT) using Superscript II reverse transcriptase (Invitrogen). qPCR was performed with the ABI 7500 fast real-time PCR system (Applied Biosystem) and SYBR Premix Ex Taq master mix (Takara).

### Immunofluorescence Staining

Frozen thymic sections were stained with Alexa Fluor 488-conjugated anti-Aire (5H12, ebioscience) and anti-keratin 14 (AF64, Covance Research) revealed with Cy3-conjugated anti-rabbit (Invitrogen). Frozen mouse and human thymic sections were stained with anti-RANK antibodies (R12-31, BioLegend for mouse detection; 80707, RnD systems for human detection) revealed with Cy3-conjugated goat anti-rat IgG (BioLegend) and with Alexa Fluor 488-conjugated donkey anti-mouse (Thermofischer) for mouse and human staining, respectively. Sections were counterstained with 1 μg/ml DAPI as previously described (59). Images were acquired with a LSM 780 Leica SP5X confocal microscope and quantified with ImageJ software.

### Statistical Analysis

Statistical significance was assessed with GraphPad Prism 6 software using Student's t test or Anova on multiple variable analyses *, P<0.05; **, P<0.01; ***, P<0.001, ****, P<0.0001. Correlations were calculated using the nonparametric Spearman correlation test. Error bars represent mean±SEM.

### Study Approval

Experiments were performed in accordance with the animal care guidelines of the European Union and French laws. All animal procedures were approved by and performed with in accordance with guidelines of the Centre d'Immunologie de Marseille-Luminy (CIML).

### Results

### RANKL is Upregulated During the Early Phase of Thymic Regeneration

Because at steady state RANKL has been reported as a potent regulator of mTEC differentiation (25, 27, 31), we investigated whether this cytokine plays a role in thymic regeneration. To this, we first analyzed RANKL expression in the thymus at day 3 after SL-TBI (d3 SL-TBI) and found that RANKL was strongly upregulated in CD45⁺hematopoietic cells compared to untreated (UT) WT mice. Among hematopoietic cells, LTi cells, identified as CD4⁺CD3⁻IL-7Rα⁺RORγt⁺, and CD4⁺ SP cells were clearly the main producers of RANKL, which was upregulated in a radiation dose-dependent manner. To definitively confirm the cellular source of RANKL, we analyzed the thymus from Rorc^{-/-} mice, defective in LTi cells (35). Under physiological conditions, the thymus from Rorc^{-/-} mice showed similar levels of RANKL mRNA compared to WT mice. In contrast, at d3 SL-TBI, while the expression of RANKL was strongly upregulated in the WT thymus, thymus from Rorc^{-/-} mice failed to increase RANKL, suggesting that LTi cells are required for RANKL upregulation after thymic damage. However, since Rorc^{-/-} mice show reduced numbers of DP and CD4+ SP cells, we also analyzed RANKL expression in the thymus of UT in ZAP-70^{-/-} mice, lacking SP cells, to determine the contribution of CD4+ SP cells in RANKL expression. Strikingly, thymus from ZAP-70^{-/-} mice failed to increase RANKL after SL-TBI, indicating that CD4⁺ SP cells constitute the major source of RANKL. Furthermore, RANKL expression in the thymus of Rag2−/− mice, lacking both DP and CD4⁺ SP but having normal numbers of LTi cells, is upregulated after SL-TBI but to a lesser extent than in WT thymus at d3 SL-TBI. These data thus indicate that RANKL is mainly produced by CD4⁺ SP and LTi cells after irradiation. RANKL was upregulated in these two cell-types until day 10 after SL-TBI with no hematopoietic rescue, implying that RANKL upregulation occurs in the early phase of thymic regeneration.

### The Administration of RANKL Boosts TEC Regeneration Upon Thymic Damage

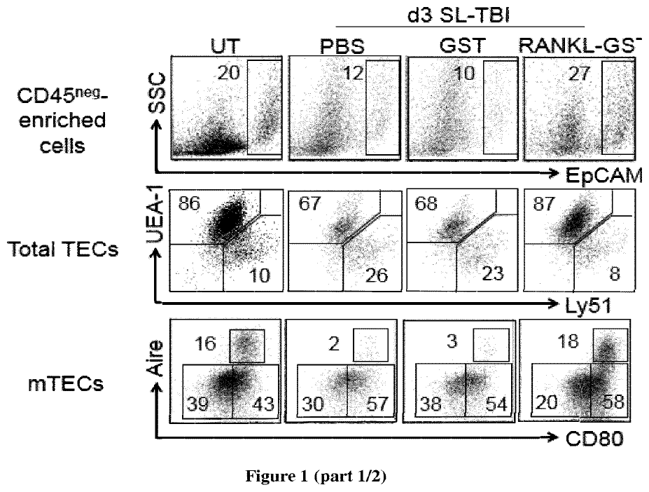

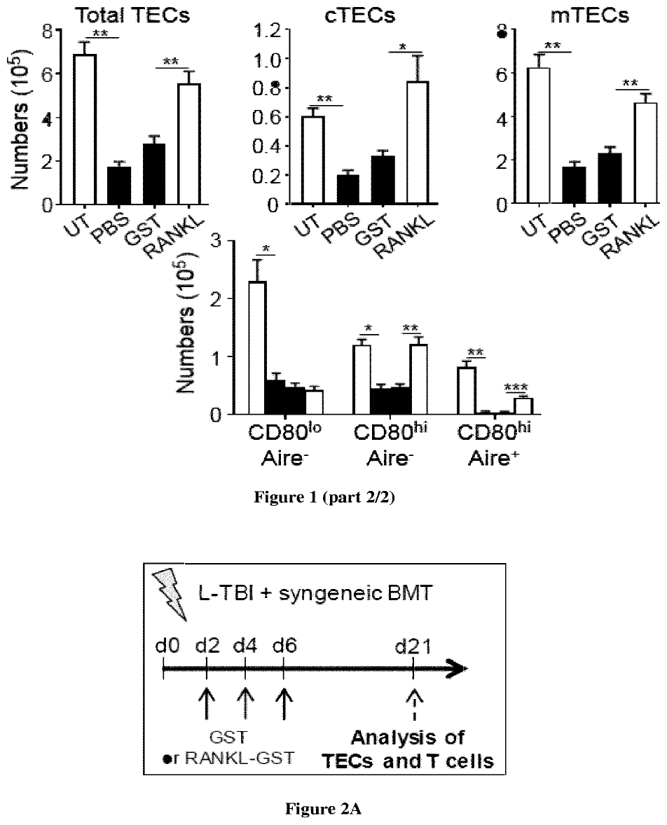

The aforementioned data strongly suggest that RANKL could play a role in thymic regeneration after irradiation. To confirm this assumption, WT mice were treated with a neutralizing anti-RANKL antibody (IK22/5) during three days after SL-TBI. PBS- and isotype antibody-treated mice were used as controls. The administration of neutralizing anti-RANKL antibody led to an impaired TEC regeneration illustrating by a 2.5-fold decrease in numbers of total TECs (EpCAM⁺), cTECs (EpCAM⁺UEA-1⁻Ly51⁺)and mTECs (EpCAM⁺UEA-1⁺Ly51⁻) compared to controls. In a therapeutic perspective, we next investigated whether conversely the ex vivo administration of RANKL protein shows beneficial effects on TEC regeneration. WT mice were treated with RANKL-GST protein during three days after SL-TBI. PBS- and GST-treated mice were used as controls. Remarkably, RANKL-treated mice showed a two-fold increase in numbers of total TECs, cTECs and mTECs compared to controls (FIG. 1). RANKL treatment also enhanced CD80hiAire−, CD80hiAire+ mTECs and several TEC subsets based on MHCII expression level (36): mTEC^{hi} (MHCII^{hi}UEA-1⁺), TEC^{lo} (MHCII^{lo}UEA-1^{lo}), and mTEC^{lo} (MHCII^{lo}UEA-1⁺). Interestingly, a TEC population described to be enriched in TEPCs defined as α6-integrin^{hi}Sca-1^{hi}MHCII^{lo} in the TEC^{lo} subset (36) was also increased.

To gain mechanistic insights into the mode of action of RANKL, we analyzed the proliferation status of cTECs, mTECs and TEPCs. Numbers of Ki-67⁺ cells in these three populations were increased in RANKL-treated mice. Purified mTECs showed reduced expression of Bax, Bid and Bak pro-apoptotic genes and cTECs reduced expression of Bax and increased expression of the Bcl-xl anti-apoptotic gene. Furthermore, the density of medullary Aire⁺ cells and the expression of Aire and Aire-dependent TRAs were enhanced. Interestingly, we also found that RANKL stimulated in cTECs the expression of Selp, ICAM-1 and CCL21, implicated in thymus homing of lymphoid progenitors. Altogether, these data show that RANKL administration boosts TEC proliferation, survival and differentiation and thus enhances TEC regeneration after thymic damage.

### RANKL Administration Induces LTα Upregulation in LTi Cells After TBI

We next investigated the underlying mechanism(s) of RANKL treatment. Interestingly, we found that irradiation led to the upregulation of RANKL cognate receptor, RANK on LTi cells. Thus, a possible mechanism is that RANKL acts on this cell-type. Furthermore, during embryogenesis, in vitro experiments have shown that RANKL induces LTα in peripheral LTi cells (37). To investigate whether RANKL regulates in vivo LTα in thymic LTi cells after irradiation, WT mice were treated with RANKL-GST or GST proteins for three days after SL-TBI. Thymic LTi cells from RANKL-treated mice upregulated LTα compared to those from GST-treated mice. Conversely, the administration of a neutralizing anti-RANKL antibody inhibited LTα upregulation in LTi cells that was expressed at similar level than that observed in non-irradiated WT mice (UT). Moreover, in vitro stimulation with RANKL-GST also significantly stimulated LTα expression specifically in LTi cells while the addition of RANKL antagonist, RANK-Fc, fully blocked LTα induction, demonstrating the specificity of the treatment used. Consistently, LTα upregulation tightly correlated with that of RANKL during the course of BMT. Interestingly, both LTα and LTβ mRNAs were increased in the total thymus at d3 SL-TBI compared to UT WT mice. LTα was specifically induced in hematopoietic cells and LTi were the main producers of LTα and LTβ. We hypothesized that LTα could be expressed as a membrane anchored LTα1β2 heterocomplex, which only binds to LTβR (38). We used a soluble LTβR-Fc fusion protein, which detects the two LTβR ligands, LTα1β2 and LIGHT. In contrast to LTα and LTβ, LIGHT was slightly expressed and not upregulated after thymic injury, indicating that LTβR-Fc staining detects only LTα1β2 in LTi cells, which is upregulated in a radiation dose-dependent manner. Finally after BMT, LTα protein was selectively upregulated in LTi cells from recipient and not from donor origin until day 6 after thymic injury, showing the importance of the host LTi cells in LTα production. These data thus show that RANKL treatment induces LTα expression in LTi cells early after thymic injury.

### LTα is Critical for TEC Regeneration and De Novo Thymopoïesis During the Course of BMT

We next addressed whether LTα upregulation in response to RANKL treatment after TBI is involved in thymic regeneration. In line with this assumption, total TECs, cTECs and mTECs but also TEPC-enriched cells upregulated LTβR at d3 SL-TBI. While at steady state, LTα^{-/-} mice, in which the expression of LTα1β2 is fully lost, did not show any defect in thymocytes and TEC subsets (32, 34), numbers of cTECs, mTECs and mTEC subsets (CD80^{lo}Aire⁻, CD80^{hi}Aire⁻ and CD80^{hi}Aire⁺) as well as the density of Aire⁺ cells were dramatically reduced at d3 SL-TBI. Of note, no significant defect neither in CD45⁻PDGFRα⁺ fibroblasts nor in thymic LTi cells was observed in LTα^{-/-} mice at d3 SL-TBI. To definitively address the role of LTα during thymic recovery after BMT, lethally irradiated CD45.2 WT or LTα^{-/-} recipients were reconstituted with CD45.1 congenic BM cells (WT CD45.1:WT or WT CD45.1:LTα^{-/-} mice) and TEC numbers were analyzed at day 10, 21 and 65 after BMT. We observed reduced numbers of total TECs, cTECs and mTECs as well as mTEC subsets in WT CD45.1:LTα^{-/-} mice compared to WT CD45.1:WT controls at all time points analyzed. Moreover, cTEC^{hi}, mTEC^{hi}, TEC^{lo}, mTEC^{lo} and TEPC-enriched cells were also reduced. Importantly, total TECs, cTECs, mTECs and TEPCs were less proliferative. A reduced density of medullary Aire⁺ cells was still detectable at d65 after BMT and consequently, the expression of Aire-dependent TRAs (SP1 and SP2) was strongly affected. The expression of an Aire-independent TRA (casein β) and Fezf2 as well as its target genes (Apoc3, Fabp9 and Resp18) (5) were also reduced. These data thus reveal that LTα is critical for TEC regeneration including TEPCs during the course of BMT.

In line with these thymic environmental defects, thymocytes were reduced from double-negative (DN) to SP stage after BMT in WT CD45.1:LTα^{-/-} mice. Consequently, numbers of peripheral CD4⁺ and CD8⁺ T cells as well as CD4⁺Foxp3⁺ regulatory T cells (Tregs) from CD45.1 donor origin were reduced in the blood and spleen of WT CD45.1:LTα^{-/-} mice from d21 to d100 after BMT.

Since de novo thymopoïesis was impaired from DN1 stage in WT CD45.1:LTα^{-/-} mice, we analyzed early T-lineage progenitors (ETPs; CD4⁻CD8⁻CD44⁺CD25⁻Lin⁻CD117⁺). Whereas numbers of ETPs were normal in LTα^{-/-} thymus at steady state, ETPs from CD45.1 donor origin were reduced in WT CD45.1:LTα^{-/-} chimeras until two months after BMT. This defect was not attributable to impaired hematopoietic progenitors because normal numbers of prethymic progenitors were observed in the BM of these mice. We hypothesized that reduced ETPs could be due to a reduced homing capacity of circulating T-cell progenitors. Thymus homing is controlled by a multistep adhesion cascade initiated by P-selectin slowing down T-cell progenitors and allowing them to respond to CCL25, CCL21/19 gradients and to engage with ICAM-1 and VCAM-1 expressed by the thymic stroma, leading to a firm arrest (39, 42). Strikingly, we found that purified CD31+ endothelial cells from WT CD45.1:LTα^{-/-} mice showed a reduced expression of adhesion molecules such as ICAM-1, VCAM-1 and P-selectin at d21 after BMT. Furthermore, purified EpCAM+ TECs in these mice also showed a reduced expression of CCL19 and CCL21. Endothelial cells and TECs were thus defective in key molecules involved in thymus homing in absence of LTα during BMT. To firmly demonstrate that thymus homing of T-cell progenitors was altered in LTα^{-/-} mice, short-term homing assays were performed by injecting CD45.1 congenic BM cells into irradiated WT and LTα^{-/-} recipients. LTα^{-/-} thymi imported three-fold less ETPs than WT thymi after thymic injury.

It is noteworthy that the effects of LTα on TEC and T-cell recovery were independent of those mediated by IL-23-regulated IL-22 described to be involved in thymic regeneration (43) since LTα^{-/-} mice exhibited similar production of these two cytokines after TBI. Of note, RANKL was expressed at normal level at d3 SL-TBI in LTα^{-/-} mice, suggesting that LTα did not regulate RANKL. Altogether, these results reveal that RANKL-regulated LTα constitutes an indispensable pathway for thymic regeneration.

We next investigated the respective efficiency of IL-22 and RANKL in thymic recovery during BMT. BM-transplanted mice were treated with IL-22 or RANKL at d2, d4, and d6 after BMT and thymic regeneration was analyzed at d21 (data not shown). We found that IL-22 and RANKL administrated alone increased similarly numbers of developing T cells including ETPs and ameliorated peripheral T-cell reconstitution. Importantly, IL-22 alone increased only numbers of CD80^{hi} mTECs, whereas RANKL alone enhanced numbers of all TEC subsets including cTECs and mTECs from the immature CD80^{lo} to the mature CD80^{hi} stage. The numbers of cTEC^{hi}, mTEC^{hi}, TEC^{lo}, mTEC^{lo}, and TEPC-enriched cells were also increased only in RANKL-treated mice. Thus, RANKL and IL-22 do not exhibit the same effects on TEC regeneration with a preferential effect for IL-22 on only mTEC^{hi} and a more large effect for RANKL on all TEC subsets. These data thus indicate that RANKL shows a superior ability than IL-22 to recover TECs after BMT.

### RANKL Administration Enhances Thymic Regeneration Upon BMT in an LTα-Dependent Manner

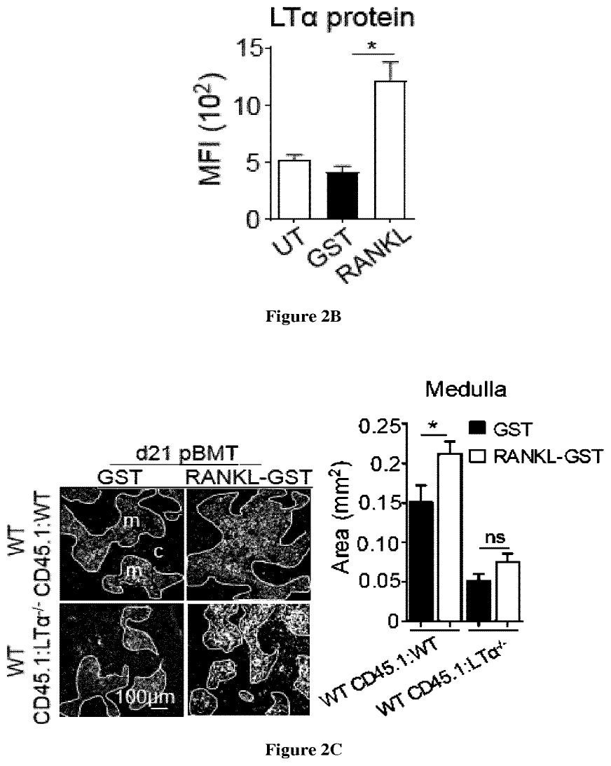

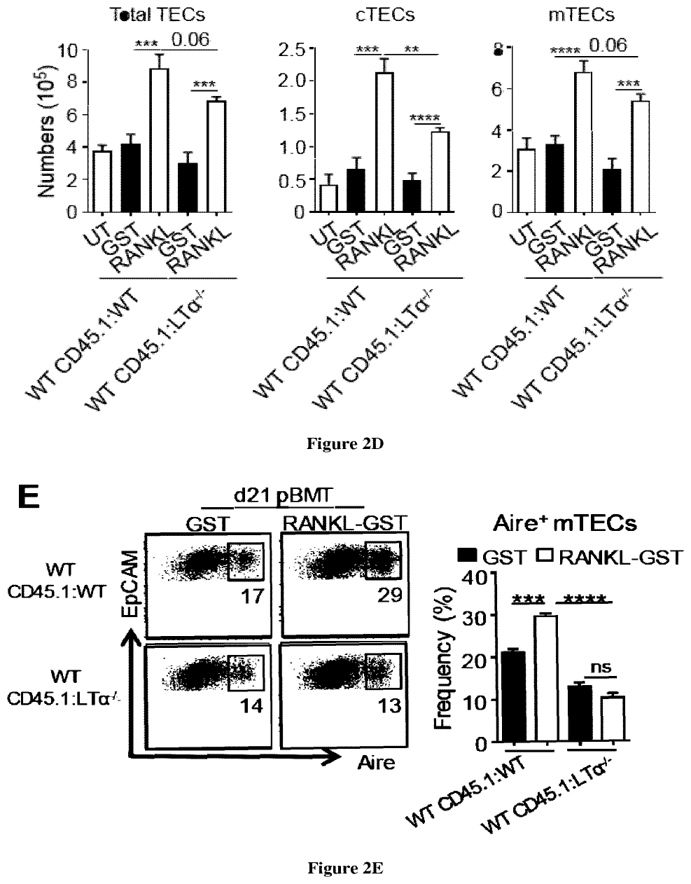

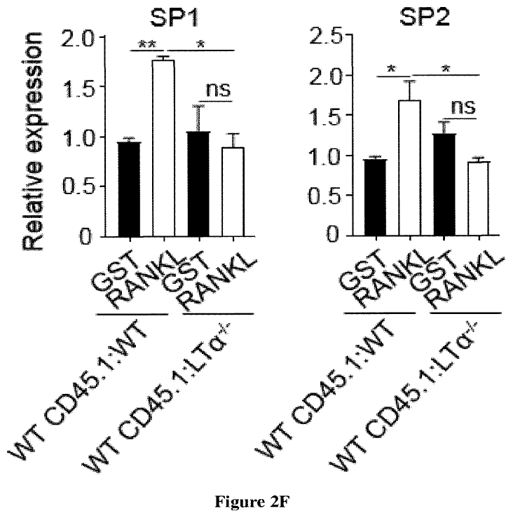

Since RANKL treatment improves TEC regeneration upon SL-TBI (FIG. 1), we next evaluated whether RANKL boosts thymic recovery during the course of BMT. WT mice transplanted with CD45.1 BM cells were treated with RANKL-GST or GST at d2, d4 and d6 after BMT and thymic regeneration was analyzed at d21 (FIG. 2A) and d65 after BMT. In these experiments, as observed at d3 SL-TBI, RANKL treatment also upregulated LTα in LTi cells, which is still detectable at d21 pBMT (FIG. 2B). RANKL-treated WT CD45.1:WT mice showed increased medullary areas, numbers of TEC subsets, Aire⁺ mTEC frequency and Aire-dependent TRAs compared to GST-treated mice (FIG. 2C-F). RANKL also increased numbers of endothelial cells. Because we found that RANKL regulates LTα, we next investigated whether these beneficial effects on TECs mediated by RANKL require LTα expression. The administration of RANKL in WT CD45.1:LTα^{-/-} mice increased TEC numbers but to a lesser extent compared to RANKL-treated WT CD45.1:WT mice (FIG. 2D). In contrast, RANKL treatment in these mice did not enhance neither Aire⁺ mTEC frequency nor Aire-dependent TRAs, indicating that LTα is critical for regeneration of Aire⁺ mTECs (FIG. 2E-F).

Interestingly, RANKL in WT CD45.1:WT chimeras substantially increased numbers of total donor cells and thymocytes of CD45.1 origin from ETP to SP stages at d21 and d65 upon BMT. In contrast, RANKL administration in WT CD45.1:LTα^{-/-} mice had a poor effect on de novo thymopoïesis. To decipher the mode of action of RANKL on T-cell reconstitution, we performed short-term homing assays in irradiated WT and LTα^{-/-} mice treated with GST or RANKL. Strikingly, the receptivity capacity of circulating progenitors was substantially enhanced in RANKL-treated WT CD45.1:WT mice compared to GST-treated controls. Importantly, this was not due to increased numbers of prethymic progenitors in the BM upon RANKL treatment. In contrast, RANKL had no effect on ETP homing in WT CD45.1:LTα^{-/-} mice. Consequently, RANKL treatment increased peripheral T-cell reconstitution only in WT CD45.1:WT mice after BMT. Altogether, these data demonstrate that LTα is critical for optimal effects of RANKL administration on TEC regeneration, thymus homing of lymphoid progenitors and T-cell reconstitution upon BMT.

### Bisphosphonate Treatment Protects From Bone Resorption Without Affecting RANKL-Improved Thymic Regeneration

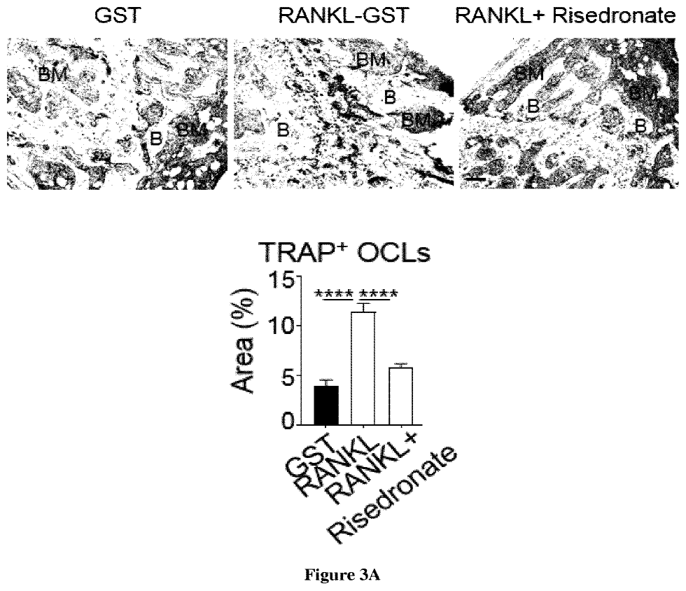

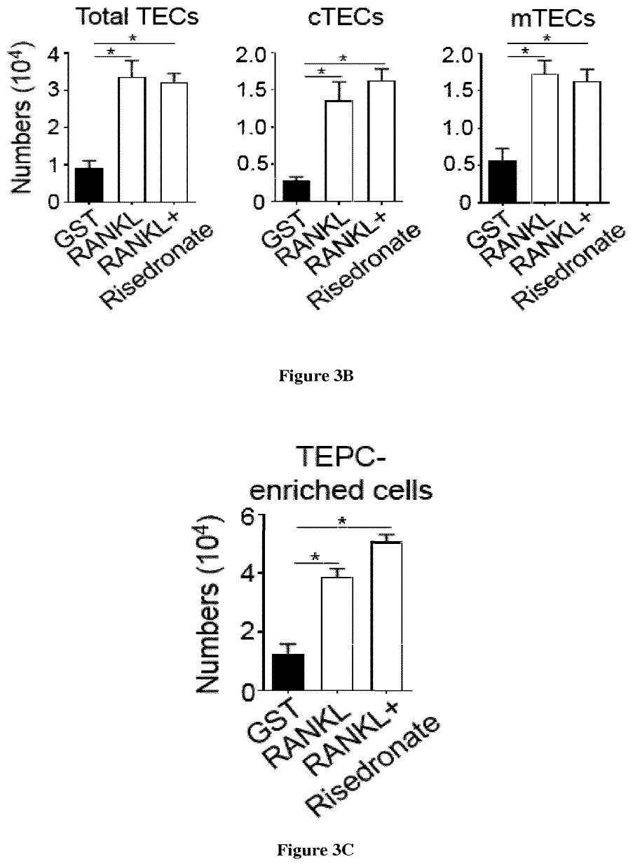

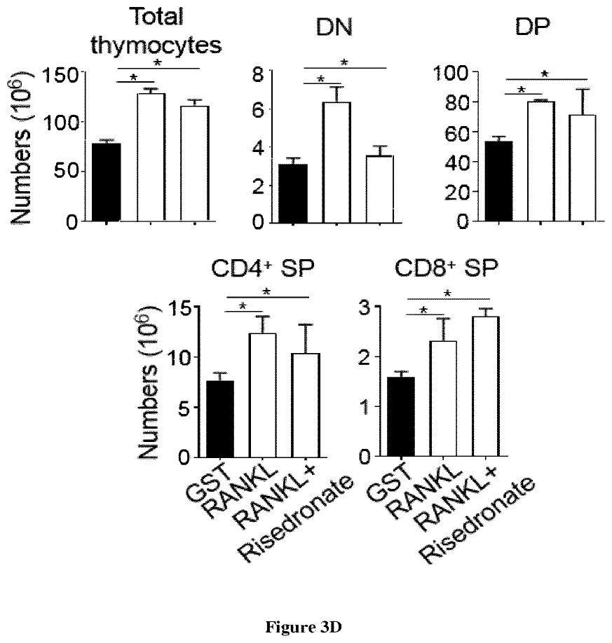

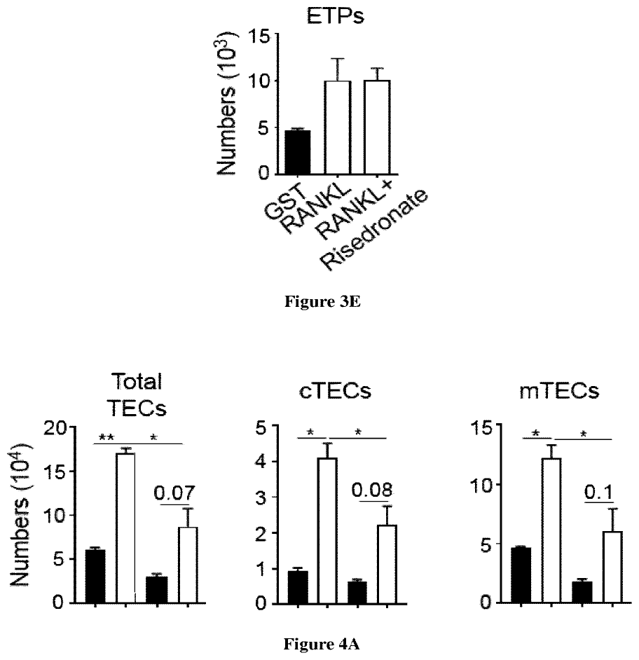

Given that RANKL administration upon BMT increased primitive progenitors in the BM, which correlated with the development of active osteoclasts (Kollet O, Dar A, Shivtiel S, Kalinkovich A, Lapid K, Sztainberg Y, Tesio M, Samstein R M, Goichberg P, Spiegel A, Elson A, Lapidot T. (2006) Osteoclasts degrade endosteal components and promote mobilization of hematopoictic progenitor cells. Nat Med. 12(6):657-64), we have investigated whether combining RANKL with bisphosphonates, which is described to prevent bone resorption (Tomimori Y, Mori K, Koide M, Nakamichi Y, Ninomiya T, Udagawa N, Yasuda H. (2009) Evaluation of pharmaceuticals with a novel 50-hour animal model of bone loss. J Bone Miner Res. 2009 July; 24(7):1194-205), protects from the development of osteoclasts without interfering with RANKL beneficial effects on thymic regeneration during BMT. The development of osteoclasts and thymic regeneration were analyzed in WT mice treated with GST and RANKL as well as in mice co-treated with risedronate and RANKL. Active osteoclasts were identified by expression of the phosphatase TRAP 21 days after BMT. The areas stained for TRAP⁺ osteoclasts were increased in RANKL-treated mice compared to GST-treated mice (FIG. 3A). Strikingly, TRAP⁺ areas in mice co-treated with risedronate and RANKL returned at the same level than in GST-treated mice. Importantly, this combined treatment did not impair TEC regeneration and T-cell reconstitution mediated by RANKL (FIG. 3B-F), indicating that risedronate can be used concomitantly with RANKL to protect from bone resorption.

### RANKL Treatment has Also Beneficial Effects on Thymic Recovery Upon BMT in Aged Individuals

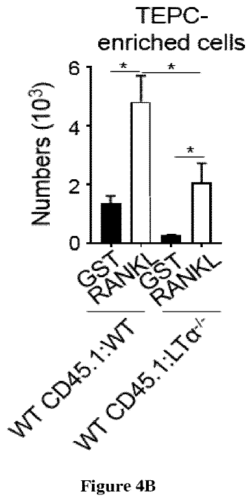

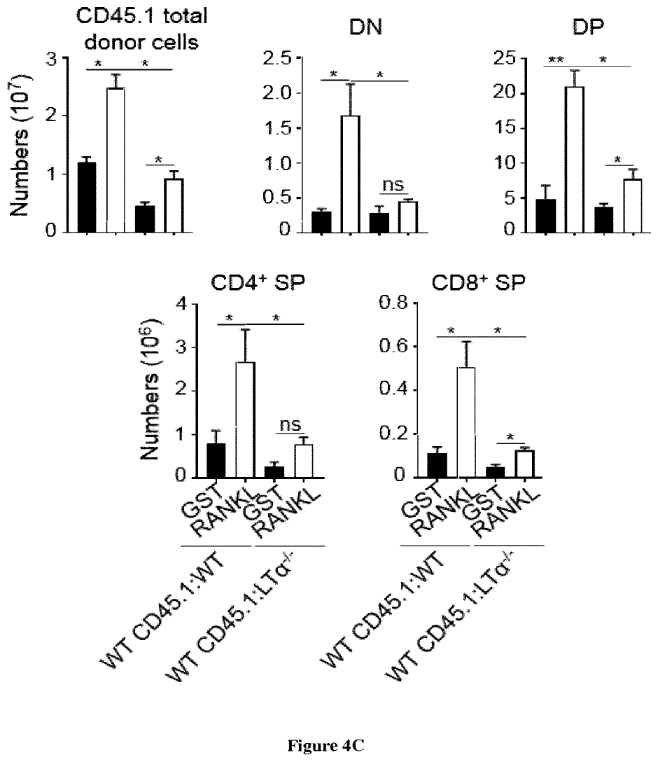

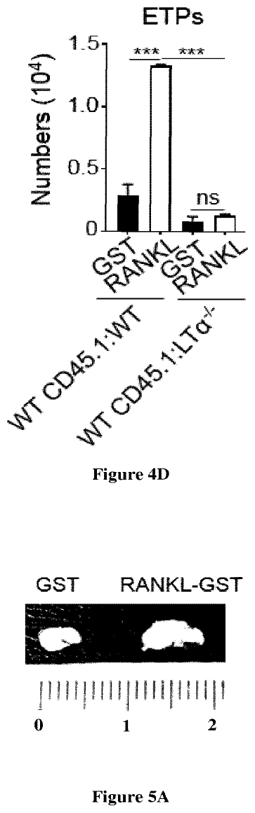

Because the recovery of T-cell functions upon BMT is known to be delayed and less efficient in elderly patients compared to young individuals (44), we finally investigated whether RANKL beneficial effects are persistent with age. To this, WT mice of 6-8 months, in which early thymic involution is characterized by a decline in TEC cellularity (45, 46), were subjected to the same treatment described in FIG. 2A. We found that RANKL increased numbers of cTECs, mTECs and TEPCs (FIG. 4A-B). All thymocytes including ETPs were also increased in these mice (FIG. 4C-D). Importantly, RANKL administration in WT CD45.1:LTα^{-/-} chimeras did not improve significantly TEC cellularity compared to RANKL-treated WT CD45.1:WT mice (FIG. 4A-B). Moreover, RANKL treatment had only minor effects on de novo thymopoïesis in WT CD45.1:LTα^{-/-} chimeras (FIG. 4C-D). Peripheral T-cell reconstitution was thus only enhanced in RANKL-treated WT CD45.1:WT mice. This set of data is consistent with the fact that LTi cells persisted with age and upregulated LTα1β2 after TBI in older mice. Furthermore, BM-transplanted LTα^{-/-} mice of 6-8 months of age showed defective TEC regeneration, de novo thymopoiesis and peripheral T-cell reconstitution. Altogether, our data indicate that RANKL treatment boosts thymic recovery after BMT not only in young but also in older individuals in an LTα-dependent manner.

### The Administration of RANKL Reverses the Effects of Thymic Involution

Age-associated thymic involution results in decreased TEC cellularity and T-cell development, which leads to increased morbidity and mortality in a numerous clinical settings. Importantly, the thymus shows compelling plastic properties and thus the effects of thymic involution can be therapeutically reversed. Since we found that RANKL improve thymic regeneration upon BMT in young and aged individuals, we hypothesized that this treatment could increase thymopoietic capacity at steady state in aged mice to ameliorate the effects of age-associated thymic involution. 12 months of age WT mice were treated with GST or RANKL-GST during three consecutive days and thymus composition was analyzed 21 days later. Strikingly, the thymus size was increased in RANKL-treated mice compared to GST-treated mice (FIG. 5A). Furthermore, numbers of total TECs, cTECs and mTECs as well as TEPC-enriched cells were substantially increased in these mice (FIG. 5B, C). T cell cellularity in RANKL-treated aged mice increased about 2.5 fold compared to GST controls (FIG. 5D,E). Importantly, although numbers of T-cell subsets in RANKL-treated aged mice did not recover to the same level than those observed in young mice, numbers of TECs and ETPs were barely similar to mice of 6 weeks of age. Finally, RANKL treatment in mice of 12 months of age increased peripheral T-cell reconstitution at a similar level than that observed in young mice (data not shown). Thus, these data show that RANKL partially reverses the effects of thymic involution.

### The Expression of RANKL Receptor, RANK, is Conserved in the Thymic Medulla in Human

To determine whether the administration of RANKL protein could be conceivable in human, we first compared the expression of its receptor, RANK, on mouse and human thymic sections. Similarly to the mouse thymus, RANK was mainly expressed in the thymic medulla in human (FIG. 6). These data suggest that thymic human cells should be able to respond to RANKL stimulation and thus that this treatment could have significant clinical impacts for improving thymus reconstitution during BMT as well as reversing the effects of age-associated thymic involution.

### Discussion

Pre-BMT conditioning induces severe damages on the thymic microenvironment, which results in delayed lymphocyte production. It is therefore of paramount clinical interest to discover new molecules that enhance thymic regeneration for an efficient recovery of the immune system (8, 11).

Our study demonstrates that the administration of RANKL substantially improves thymic recovery during BMT. We found that thymic LTi cells and CD4⁺ SP cells, described to be radio-resistant (43) (Tomoo Ueno et al. J Exp Med, 2004), constitute the major source of RANKL. Administration of RANKL induces LTa expression specifically in LTi cells after injury. Furthermore, thymic LTi cells from recipient origin upregulated both RANKL and LTα during the early phase of BMT. Thus, these cells change their phenotype upon stress-induced thymic damage. Thymic LTi cells are thus likely in a “quiescent stage” at steady state and are activated after irradiation to repair the injured tissue. When considering that LTi cells expressing RANKL and LTα are involved in the organogenesis of lymph nodes (47), our data suggest that thymic LTi cells likely reactivate an embryonic program to repair the thymus after irradiation.

Interestingly, we found that RANKL administration after TBI boosts the regeneration of TEC subsets including TEPC-enriched cells. Conversely, the administration of a neutralizing anti-RANKL antibody leads to an impaired TEC regeneration. Furthermore, flow cytometry, histology and qPCR experiments indicated that RANKL administration also boosts the regeneration of Aire⁺ mTECs. Importantly, RANKL treatment during the early phase of BMT enhances numbers of TECs, ETPs and thymocytes. We demonstrated that thymus homing of T-cell progenitors and T-cell output are improved. Notably, although mice were treated during the early phase of BMT, RANKL treatment had long-term beneficial effects detectable until two months after BMT on both TEC and T-cell compartments. Improved T-cell reconstitution can be explained by increased stromal niches linked to increased TEC cellularity but also to enhanced thymus homing of lymphoid progenitors, which is a critical step for ameliorating T-cell recovery (19, 21). The latter effect is likely mediated by increased expression of adhesion molecules and chemokines involved in this process. Nevertheless, we cannot exclude that the enhanced thymus homing by RANKL administration is also favored by increased vasculature, which is important for thymus homing. Thus, our data reveal that RANKL plays distinct roles in the thymus at steady state and during BMT.

We further found by in vitro and in vivo stimulations that RANKL induces LTα in LTi cells, which express its cognate receptor, RANK. Importantly, the administration of a neutralizing RANKL-antibody inhibits LTα upregulation in LTi cells, indicating that RANKL specifically controls LTα upregulation after SL-TBI. In contrast, LTα did not regulate RANKL, indicating that LTα acts downstream of RANKL. Whereas LTα is dispensable at steady state for TEC and T-cell cellularity, we found that LTα is critical for the recovery of thymic function. TEC subsets including TEPCs were severely reduced in LTα^{-/-}-transplanted recipients from up to two months after BMT. Moreover, all thymocyte subsets as well as ETPs were also reduced in these mice likely due to defective thymus homing capacity. In accordance with our data, it has been recently reported that LTβR regulates VCAM-1 and ICAM-1 on endothelial cells, known to promote T-cell progenitor entry in the thymus (40, 49, 50). Consistently, we found that LTα expression is important for the expression of adhesion molecules and chemokines in stromal cells during BMT. Given that in the steady state thymus, LTα expressed by SP thymocytes, is involved in the regular thymic architecture (12, 33, 51, 52), our data show that LTα expressed by LTi cells after thymic injury plays distinct roles.

Furthermore, it has been described that IL-22 participates to thymus recovery (43). Our data reveal that RANKL-regulated LTα in LTi cells represents a distinct mechanism of that mediated by IL-22 in thymic regeneration and thus highlighting that this cell-type uses different mechanisms for thymic repair. Importantly, although IL-22 treatment during BMT enhances only mature CD80^{hi} mTECs, our data demonstrate that RANKL treatment ameliorates the regeneration of not only CD80^{hi} mTECs but also CD80^{lo} mTECs and cTECs. The administration of RANKL boosts also the recovery of TEPCs, which are essential for the renewal of stromal niches after BMT. These results thus show that in contrast to IL-22, RANKL has large spectrum effects on TECs with a superior ability to regenerate these cell populations. Importantly, whereas IL-22 treatment had no more effect on thymopoiesis at d28 after BMT, RANKL administration boosts thymic regeneration until d65 after BMT. Thus, RANKL constitutes an innovative therapy to enhance thymic regeneration after BMT by acting on both TEC and T-cell reconstitution. One would expect that thymic regeneration during the course of BMT is also defective in RANKL^{-/-} mice. Unfortunately, we were unable to test this hypothesis since RANKL^{-/-} mice show severe growth retardations (53) and exhibit a drastic reduction in mTECs (27).

In addition, the administration of fibroblast growth factor 7 (Fgf-7) before BMT has been shown to protect TECs and ameliorates the thymopoïctic capacity (Min D, Taylor P A, Panoskaltsis-Mortari A, Chung B, Danilenko D M, Farrell C, Lacey D L, Blazar B R, Weinberg K I. (2002) Protection from thymic epithelial cell injury by keratinocyte growth factor: a new approach to improve thymic and peripheral T-cell reconstitution after bone marrow transplantation. Blood. 15; 99(12):4592-600). However, in contrast to RANKL, Fgf-7 shows a protective but not a regenerative effect during BMT.

Nevertheless, we demonstrate that RANKL administration has beneficial effects on thymic recovery. Moreover, RANKL treatment in BM-transplanted LTα^{-/-} mice had only minor effects on TEC regeneration, numbers of ETPs and strikingly de novo thymopoiesis was not ameliorated. Interestingly, whereas LTα is dispensable for Aire⁺ mTEC differentiation at steady state (32), the regeneration of these cells induced by RANKL treatment critically depends on LTα. These data indicate that the mechanisms involved in Aire⁺ mTEC regeneration are distinguishable from those implicated in their emergence/differentiation at steady state. Importantly, beneficial effects of RANKL treatment observed on TEC regeneration are likely not due to a direct action on TECs because the effects of RANKL on these cells were modest in absence of LTα. Thus, these data argue in favor of model in which RANKL acts indirectly on TECs through LTi cells that express LTα. This notion is supported by the fact that the turnover rate of mTECs is of around 2 weeks (54, 55) and thus it is unlikely that RANKL injected early after BMT still acts on mTECs two months later.

To avoid any potential side effects of the systemic administration of RANKL, such as osteoporosis, a possible strategy would be to deliver directly this molecule intrathymically in patients after cytoablative conditioning or combined RANKL with bisphosphonate to prevent bone resorption (57). The administration of bisphosphonate leads to a decrease in osteoclast differentiation and in apoptosis of mature osteoclasts. Importantly, this drug that inhibits bone resorption does not affect RANKL signaling (Kim Y H, Kim G S, Jeong-Hwa B (2002) Inhibitory action of bisphosphonates on bone resorption does not involve the regulation of RANKL and OPG expression. Exp Mol Med 34: 145-151; Verde M E, Bermejo D, Gruppi A, Grenon M (2015) Effect of Bisphosphonates on the Levels of Rankl and Opg in Gingival Crevicular Fluid of Patients With Periodontal Disease and Post-menopausal Osteoporosis. Acta Odontol Latinoam 28: 215-221). Furthermore, bisphosphonate combined with RANKL have been shown to suppress mouse and human osteoclast differentiation mediated by RANKL treatment (Tomimori Y, Mori K, Koide M, Nakamichi Y, Ninomiya T, Udagawa N, Yasuda H (2009) Evaluation of pharmaceuticals with a novel 50-hour animal model of bone loss. J Bone Miner Res 24: 1194-1205). Our data demonstrate that a co-treatment of risedronate and RANKL during BMT protects efficiently from bone resorption without interfering with RANKL beneficial effects on thymic regeneration. Thus, RANKL treatment in clinic is expected to be promising for enhancing the regeneration of immune functions in patients whose thymus has been severely damaged.

Interestingly, RANKL administration is also efficient for TEC and T-cell regeneration during BMT in older individuals in which thymic involution results in diminished TEC cellularity, disrupted thymic architecture and decreased T-cell output (45, 46). These results are of special interest for elderly patients in which the recovery of T-cell functions upon BMT is less efficient (44).

Age-induced thymic involution results in a reduced efficacy of the immune system to fight against opportunistic infections and also favors the development of autoimmunity and increases the incidence of cancer (Lynch H E, Goldberg G L, Chidgey A, Van den Brink M R, Boyd R, Sempowski G D (2009) Thymic involution and immune reconstitution. Trends Immunol. July; 30(7):366-73). Thymic function declines in the second year of life and is marked by a progressive diminution mainly in cTECs but also in mTECs (Steinmann G G, Klaus B, Müller-Hermelink H K (1985) The involution of the ageing human thymic epithelium is independent of puberty. A morphometric study. Scand J Immunol.; 22(5):563-75). The administration of insulin growth factor (IGF) or Ghrelin peptide hormone partly improves thymic compartments in aged mice (Montecino-Rodriguez El, Clark R, Dorshkind K. (1998) Effects of insulin-like growth factor administration and bone marrow transplantation on thymopoiesis in aged mice. Endocrinology. 139(10):4120-6; Dixit V D, Yang H, Sun Y, Weeraratna A T, Youm Y H, Smith R G, Taub D D. (2007) Ghrelin promotes thymopoiesis during aging. J Clin Invest. 117(10):2778-90). Fgf-7 plays also significant roles in the correction of thymus senescence in mice (Min Dl, Panoskaltsis-Mortari A, Kuro-O M, Holländer G A, Blazar B R, Weinberg K I. (2007) Sustained thymopoiesis and improvement in functional immunity induced by exogenous KGF administration in murine models of aging. Blood. 5; 109(6):2529-37). However, the efficacy of Fgf-7 alone in clinical trials remains to be established. Our data demonstrate that a single three days course of RANKL in aged mice partially recovered the numbers of thymocytes until three weeks after treatment. Importantly, this treatment totally reversed the diminution of cTECs and mTECs but also ETPs observed upon thymic involution.

Interestingly, we show that the expression of RANK is conserved in the thymic medulla in human, indicating that RANKL treatment could be also beneficial to restore thymic function in different clinical settings.

This study thus reveals that administration of RANKL offers an innovative therapeutic strategy to boost thymic recovery at several levels: TEC regeneration, thymus homing of T-cell progenitors and de novo thymopoïesis.

### REFERENCES

In Example 1 and the preceding disclosure, various references describe the state of the art to which this invention pertains. The disclosures of these references are hereby incorporated by reference into the present disclosure:
1. Anderson, G., and Takahama, Y. 2012. Thymic epithelial cells: working class heroes for T cell development and repertoire selection. Trends in immunology 33:256-263.
2. Derbinski, J., Schulte, A., Kyewski, B., and Klein, L. 2001. Promiscuous gene expression in medullary thymic epithelial cells mirrors the peripheral self. Nat Immunol 2:1032-1039.
3. Sansom, S.N., Shikama-Dorn, N., Zhanybekova, S., Nusspaumer, G., Macaulay, I.C., Deadman, M.E., Heger, A., Ponting, C.P., and Hollander, G.A. 2014. Population and single-cell genomics reveal the Aire dependency, relief from Polycomb silencing, and distribution of self-antigen expression in thymic epithelia. Genome research.
4. Anderson, M.S., Venanzi, E.S., Klein, L., Chen, Z., Berzins, S.P., Turley, S.J., von Bochmer, H., Bronson, R., Dierich, A., Benoist, C., et al. 2002. Projection of an immunological self shadow within the thymus by the aire protein. Science 298:1395-1401.
5. Takaba, H., Morishita, Y., Tomofuji, Y., Danks, L., Nitta, T., Komatsu, N., Kodama, T., and Takayanagi, H. 2015. Fezf2 Orchestrates a Thymic Program of Self-Antigen Expression for Immune Tolerance. Cell 163:975-987.
6. Lopes, N., Serge, A., Ferrier, P., and Irla, M. 2015. Thymic Crosstalk Coordinates Medulla Organization and T-Cell Tolerance Induction. Frontiers in immunology 6:365.
7. van Ewijk, W., Shores, E.W., and Singer, A. 1994. Crosstalk in the mouse thymus. Immunol Today 15:214-217.
8. van den Brink, M.R., Alpdogan, O., and Boyd, R.L. 2004. Strategies to enhance T-cell reconstitution in immunocompromised patients. Nature reviews. Immunology 4:856-867.
9. Fletcher, A.L., Lowen, T.E., Sakkal, S., Reiseger, J.J., Hammett, M.V., Seach, N., Scott, H.S., Boyd, R.L., and Chidgey, A.P. 2009. Ablation and regeneration of tolerance-inducing medullary thymic epithelial cells after cyclosporine, cyclophosphamide, and dexamethasone treatment. Journal of immunology 183:823-831.
10. Adkins, B., Gandour, D., Strober, S., and Weissman, I. 1988. Total lymphoid irradiation leads to transient depletion of the mouse thymic medulla and persistent abnormalities among medullary stromal cells. Journal of immunology 140:3373-3379.
11. Hollander, G.A., Krenger, W., and Blazar, B.R. 2010. Emerging strategies to boost thymic function. Current opinion in pharmacology 10:443-453.
12. Irla, M., Guenot, J., Scaly, G., Reith, W., Imhof, B.A., and Serge, A. 2013. Three-dimensional visualization of the mouse thymus organization in health and immunodeficiency. Journal of immunology 190:586-596.
13. Irifune, T., Tamechika, M., Adachi, Y., Tokuda, N., Sawada, T., and Fukumoto, T. 2004. Morphological and immunohistochemical changes to thymic epithelial cells in the irradiated and recovering rat thymus. Archives of histology and cytology 67:149-158.
14. Hakim, F.T., Cepeda, R., Kaimei, S., Mackall, C.L., McAtee, N., Zujewski, J., Cowan, K., and Gress, R.E. 1997. Constraints on CD4 recovery postchemotherapy in adults: thymic insufficiency and apoptotic decline of expanded peripheral CD4 cells. Blood 90:3789-3798.
15. Small, T.N., Papadopoulos, E.B., Boulad, F., Black, P., Castro-Malaspina, H., Childs, B.H., Collins, N., Gillio, A., George, D., Jakubowski, A., et al. 1999. Comparison of immune reconstitution after unrelated and related T-cell-depleted bone marrow transplantation: effect of patient age and donor leukocyte infusions. Blood 93:467-480.
16. King, C., Ilic, A., Koelsch, K., and Sarvetnick, N. 2004. Homeostatic expansion of T cells during immune insufficiency generates autoimmunity. Cell 117:265-277.
17. Parkman, R., Cohen, G., Carter, S.L., Weinberg, K.I., Masinsin, B., Guinan, E., Kurtzberg, J., Wagner, J.E., and Kernan, N.A. 2006. Successful immune reconstitution decreases leukemic relapse and improves survival in recipients of unrelated cord blood transplantation. Biology of blood and marrow transplantation: journal of the American Society for Blood and Marrow Transplantation 12:919-927.
18. Curtis, R.E., Rowlings, P.A., Deeg, H.J., Shriner, D.A., Socie, G., Travis, L.B., Horowitz, M.M., Witherspoon, R.P., Hoover, R.N., Sobocinski, K.A., et al. 1997. Solid cancers after bone marrow transplantation. The New England journal of medicine 336:897-904.
19. Penit, C., and Ezine, S. 1989. Cell proliferation and thymocyte subset reconstitution in sublethally irradiated mice: compared kinetics of endogenous and intrathymically transferred progenitors. Proceedings of the National Academy of Sciences of the United States of America 86:5547-5551.
20. Zlotoff, D.A., Zhang, S.L., De Obaldia, M.E., Hess, P.R., Todd, S.P., Logan, T.D., and Bhandoola, A. 2011. Delivery of progenitors to the thymus limits T-lineage reconstitution after bone marrow transplantation. Blood 118:1962-1970.
21. Chen, B.J., Cui, X., Sempowski, G.D., Domen, J., and Chao, N.J. 2004.Hematopoietic stem cell dose correlates with the speed of immune reconstitution after stem cell transplantation. Blood 103:4344-4352.
22. Fata, J.E., Kong, Y.Y., Li, J., Sasaki, T., Irie-Sasaki, J., Moorehead, R.A., Elliott, R., Scully, S., Voura, E.B., Lacey, D.L., et al. 2000. The osteoclast differentiation factor osteoprotegerin-ligand is essential for mammary gland development. Cell 103:41-50.
23. Duheron, V., Hess, E., Duval, M., Decossas, M., Castaneda, B., Klopper, J.E., Amoasii, L., Barbaroux, J.B., Williams, I.R., Yagita, H., et al. 2011. Receptor activator of NF-kappaB (RANK) stimulates the proliferation of epithelial cells of the epidermo-pilosebaceous unit. Proceedings of the National Academy of Sciences of the United States of America 108:5342-5347.
24. Knoop, K.A., Kumar, N., Butler, B.R., Sakthivel, S.K., Taylor, R.T., Nochi, T., Akiba, H., Yagita, H., Kiyono, H., and Williams, I.R. 2009. RANKL is necessary and sufficient to initiate development of antigen-sampling M cells in the intestinal epithelium. Journal of immunology 183:5738-5747.
25. Rossi, S.W., Kim, M.Y., Leibbrandt, A., Parnell, S.M., Jenkinson, W.E., Glanville, S.H., McConnell, F.M., Scott, H.S., Penninger, J.M., Jenkinson, E.J., et al. 2007. RANK signals from CD4(+)3(−) inducer cells regulate development of Aire-expressing epithelial cells in the thymic medulla. J Exp Med 204:1267-1272.
26. Roberts, N.A., White, A.J., Jenkinson, W.E., Turchinovich, G., Nakamura, K., Withers, D.R., McConnell, F.M., Desanti, G.E., Benezech, C., Parnell, S.M., et al. 2012. Rank signaling links the development of invariant gammadelta T cell progenitors and Aire(+) medullary epithelium. Immunity 36:427-437.
27. Hikosaka, Y., Nitta, T., Ohigashi, I., Yano, K., Ishimaru, N., Hayashi, Y., Matsumoto, M., Matsuo, K., Penninger, J.M., Takayanagi, H., et al. 2008. The cytokine RANKL produced by positively selected thymocytes fosters medullary thymic epithelial cells that express autoimmune regulator. Immunity 29:438-450.
28. Akiyama, T., Shimo, Y., Yanai, H., Qin, J., Ohshima, D., Maruyama, Y., Asaumi, Y., Kitazawa, J., Takayanagi, H., Penninger, J.M., et al. 2008. The tumor necrosis factor family receptors RANK and CD40 cooperatively establish the thymic medullary microenvironment and self-tolerance. Immunity 29:423-437.
29. Irla, M., Hugues, S., Gill, J., Nitta, T., Hikosaka, Y., Williams, I.R., Hubert, F.X., Scott, H.S., Takahama, Y., Hollander, G.A., et al. 2008. Autoantigen-specific interactions with CD4+ thymocytes control mature medullary thymic epithelial cell cellularity.Immunity 29:451-463.
30. Irla, M., Hollander, G., and Reith, W. 2010. Control of central self-tolerance induction by autoreactive CD4+ thymocytes. Trends in immunology 31:71-79.
31. Ohigashi, I., Nitta, T., Lkhagvasuren, E., Yasuda, H., and Takahama, Y. 2011. Effects of RANKL on the thymic medulla. European journal of immunology 41:1822-1827.
32. Venanzi, E.S., Gray, D.H., Benoist, C., and Mathis, D. 2007. Lymphotoxin pathway and Aire influences on thymic medullary epithelial cells are unconnected. J Immunol 179:5693-5700.
33. Seach, N., Ueno, T., Fletcher, A.L., Lowen, T., Mattesich, M., Engwerda, C.R., Scott, H.S., Ware, C.F., Chidgey, A.P., Gray, D.H., et al. 2008. The lymphotoxin pathway regulates Aire-independent expression of ectopic genes and chemokines in thymic stromal cells. J Immunol 180:5384-5392.
34. De Togni, P., Goellner, J., Ruddle, N.H., Streeter, P.R., Fick, A., Mariathasan, S., Smith, S.C., Carlson, R., Shornick, L.P., Strauss-Schoenberger, J., et al. 1994. Abnormal development of peripheral lymphoid organs in mice deficient in lymphotoxin. Science 264:703-707.
35. Sun, Z., Unutmaz, D., Zou, Y.R., Sunshine, M.J., Pierani, A., Brenner-Morton, S., Mebius, R.E., and Littman, D.R. 2000. Requirement for RORgamma in thymocyte survival and lymphoid organ development. Science 288:2369-2373.
36. Wong, K., Lister, N.L., Barsanti, M., Lim, J.M., Hammett, M.V., Khong, D.M., Siatskas, C., Gray, D.H., Boyd, R.L., and Chidgey, A.P. 2014. Multilineage potential and self-renewal define an epithelial progenitor cell population in the adult thymus. Cell reports 8:1198-1209.
37. Yoshida, H., Naito, A., Inoue, J., Satoh, M., Santee-Cooper, S.M., Ware, C.F., Togawa, A., and Nishikawa, S. 2002. Different cytokines induce surface lymphotoxin-alphabeta on IL-7 receptor-alpha cells that differentially engender lymph nodes and Peyer's patches. Immunity 17:823-833.
38. Gommerman, J.L., and Browning, J.L. 2003. Lymphotoxin/light, lymphoid microenvironments and autoimmune disease. Nature reviews. Immunology 3:642-655.
39. Rossi, F.M., Corbel, S.Y., Merzaban, J.S., Carlow, D.A., Gossens, K., Duenas, J., So, L., Yi, L., and Ziltener, H.J. 2005. Recruitment of adult thymic progenitors is regulated by P-selectin and its ligand PSGL-1. Nature immunology 6:626-634.
40. Scimone, M.L., Aifantis, I., Apostolou, I., von Boehmer, H., and von Andrian, U.H. 2006. A multistep adhesion cascade for lymphoid progenitor cell homing to the thymus. Proceedings of the National Academy of Sciences of the United States of America 103:7006-7011.
41. Zlotoff, D.A., Sambandam, A., Logan, T.D., Bell, J.J., Schwarz, B.A., and Bhandoola, A. 2010. CCR7 and CCR9 together recruit hematopoietic progenitors to the adult thymus. Blood 115:1897-1905.
42. Krueger, A., Willenzon, S., Lyszkiewicz, M., Kremmer, E., and Forster, R. 2010. CC chemokine receptor 7 and 9 double-deficient hematopoietic progenitors are severely impaired in seeding the adult thymus. Blood 115:1906-1912.
43. Dudakov, J.A., Hanash, A.M., Jenq, R.R., Young, L.F., Ghosh, A., Singer, N.V., West, M.L., Smith, O.M., Holland, A.M., Tsai, J.J., et al. 2012. Interleukin-22 drives endogenous thymic regeneration in mice. Science 336:91-95.
44. Toubert, A., Glauzy, S., Douay, C., and Clave, E. 2012. Thymus and immune reconstitution after allogeneic hematopoietic stem cell transplantation in humans: never say never again. Tissue antigens 79:83-89.
45. Gray, D.H., Seach, N., Ueno, T., Milton, M.K., Liston, A., Lew, A.M., Goodnow, C.C., and Boyd, R.L. 2006. Developmental kinetics, turnover, and stimulatory capacity of thymic epithelial cells. Blood 108:3777-3785.
46. Ki, S., Park, D., Selden, H.J., Seita, J., Chung, H., Kim, J., Iyer, V.R., and Ehrlich, L.I. 2014. Global transcriptional profiling reveals distinct functions of thymic stromal subsets and age-related changes during thymic involution. Cell reports 9:402-415.
47. Yoshida, H., Honda, K., Shinkura, R., Adachi, S., Nishikawa, S., Maki, K., Ikuta, K., and Nishikawa, S.I. 1999. IL-7 receptor alpha+ CD3(−) cells in the embryonic intestine induces the organizing center of Peyer's patches. International immunology 11:643-655.
48. Lind, E.F., Prockop, S.E., Porritt, H.E., and Petrie, H.T. 2001. Mapping precursor movement through the postnatal thymus reveals specific microenvironments supporting defined stages of early lymphoid development. The Journal of experimental medicine 194:127-134.
49. Lucas, B., James, K.D., Cosway, E.J., Parnell, S.M., Tumanov, A.V., Ware, C.F., Jenkinson, W.E., and Anderson, G. 2016. Lymphotoxin beta Receptor Controls T Cell Progenitor Entry to the Thymus. Journal of immunology.
50. Shi, Y., Wu, W., Chai, Q., Li, Q., Hou, Y., Xia, H., Ren, B., Xu, H., Guo, X., Jin, C., et al. 2016. LTbetaR controls thymic portal endothelial cells for haematopoietic progenitor cell homing and T-cell regeneration. Nature communications 7:12369.
51. Boehm, T., Scheu, S., Pfeffer, K., and Bleul, C.C. 2003. Thymic medullary epithelial cell differentiation, thymocyte emigration, and the control of autoimmunity require lympho-epithelial cross talk via LTbetaR. J Exp Med 198:757-769.
52. Irla, M., Guerri, L., Guenot, J., Serge, A., Lantz, O., Liston, A., Imhof, B.A., Palmer, E., and Reith, W. 2012. Antigen recognition by autoreactive cd4(+) thymocytes drives homeostasis of the thymic medulla. PLoS One 7:e52591.
53. Kong, Y.Y., Yoshida, H., Sarosi, I., Tan, H.L., Timms, E., Capparelli, C., Morony, S., Oliveira-dos-Santos, A.J., Van, G., Itie, A., et al. 1999. OPGL is a key regulator of osteoclastogenesis, lymphocyte development and lymph-node organogenesis. Nature 397:315-323.
54. Gabler, J., Arnold, J., and Kyewski, B. 2007. Promiscuous gene expression and the developmental dynamics of medullary thymic epithelial cells. Eur J Immunol 37:3363-3372.
55. Gray, D., Abramson, J., Benoist, C., and Mathis, D. 2007. Proliferative arrest and rapid turnover of thymic epithelial cells expressing Aire. J Exp Med 204:2521-2528.
56. Chinn, I.K., Blackburn, C.C., Manley, N.R., and Sempowski, G.D. 2012. Changes in primary lymphoid organs with aging. Seminars in immunology 24:309-320.
57. Tomimori, Y., Mori, K., Koide, M., Nakamichi, Y., Ninomiya, T., Udagawa, N., and Yasuda, H. 2009. Evaluation of pharmaceuticals with a novel 50-hour animal model of bone loss. Journal of bone and mineral research: the official journal of the American Society for Bone and Mineral Research 24:1194-1205.
58. van de Pavert, S.A., Ferreira, M., Domingues, R.G., Ribeiro, H., Molenaar, R., Moreira-Santos, L., Almeida, F.F., Ibiza, S., Barbosa, I., Goverse, G., et al. 2014. Maternal retinoids control type 3 innate lymphoid cells and set the offspring immunity. Nature 508:123-127.
59. Serge, A., Bailly, A.L., Aurrand-Lions, M., Imhof, B.A., and Irla, M. 2015. For3D: Full Organ Reconstruction in 3D, an Automatized Tool for Deciphering the Complexity of Lymphoid Organs. Journal of immunological methods.

### Example 2

### Introduction

A continuous T-cell production by the thymus is crucial for efficient T-cell immune responses. However, the thymus progressively declines during life, a process referred to as age-related thymic involution, which is a major cause of immunosenescence (1). This is characterized by reduced thymic mass with a disorganized cortico-medullary organization and the appearance of epithelial-free regions (2, 3). In particular, while the medulla forms a complex inter-connected structure in the young thymus, its topology is remodeled into smaller islets during aging (4, 5). Maximum thymic activity in mice is observed at 4 weeks of age, then decreases by 50% by 4 months (6, 7). In humans, thymic involution starts during the first years of life with a decrease in thymopoiesis of ˜3% per year until middle age (˜40 years old) and ˜1% per year during the rest of life (8-10). This gradual thymic decline results in a reduced naïve T-cell output and, consequently to an increased susceptibility to infections, cancers, auto-inflammatory disorders and reduced vaccine efficacy in the elderly (11-14). Importantly, a recent report revealed that mortality and risk of cancers are higher in adult patients who have undergone thymectomy, highlighting the importance of maintaining efficient thymic function with age (15).

The thymus contains stromal niches composed of highly specialized thymic epithelial cells (TEC) that provide essential cues for the proliferation, migration, differentiation, and survival of developing T cells (16). In particular, cortical TEC (cTEC) support early stages of thymopoiesis, including the recruitment of T-cell progenitors, T-cell lineage commitment and proliferation, as well as positive selection of CD4⁺ and CD8⁺ thymocytes. Via their unique ability to express thousands of tissue-restricted self-antigens, regulated by the transcription factors Aire and Fezf2, medullary TEC (mTEC) control subsequent stages of thymopoiesis, in particular the induction of self-tolerance characterized by the clonal deletion of highly autoreactive T cells and the generation of Foxp3⁺ regulatory T cells (Treg). Defects in TEC proliferation and differentiation during aging are major causes of thymic involution (7, 13, 17,18). mTEC are commonly subdivided into two main subsets: mTEC^{lo} (MHCII^{lo}CD80^{lo}) and mTEC^{hi} (MHCII^{hi}CD80^{hi}). The mTEC^{hi}/mTEC^{lo} ratio decreases during aging, suggesting a reduced antigen-presentation capacity (19). Moreover, in line with a reduced expression of tissue-restricted self-antigens, Aire expression is diminished in mTEC^{hi}, which likely contributes to an age-related increase in autoimmune disorders. Despite these major alterations, the mechanisms responsible for thymic involution remain poorly understood.

Defects in TEC are already visible in 3- and 6-month-old mice (18) and central tolerance is substantially impaired in 12-month-old (middle-aged) mice (20). Alterations in TEC composition during aging were also observed early in human (21). This suggests that thymic involution needs to be targeted at middle-age to replenish the peripheral T-cell pool and prevent the accumulation of senescent T cells. As alterations in the thymic epithelium are largely responsible for age-related thymic involution, an attractive approach to rejuvenate thymic function is to target TEC. Although the thymus is highly sensitive to aging, this organ is receptive to regenerative therapies. Preclinical studies in mice have identified potential candidates capable of regenerating TEC upon thymic injury caused by cytoreductive regimen used prior to hematopoietic cell transplantation (22-25). In the context of age-related involution, only keratin growth factor (KGF) has been reported to regenerate the thymic epithelium². However, contrary to mice, the use of a recombinant form of KGF, palifermin, in a recent clinical trial unexpectedly reduced thymopoiesis (26). Thus, the identification of therapeutic molecules capable of rejuvenating thymic function is still urgently needed. In the postnatal thymus, the bone-associated cytokine RANK ligand (RANKL) has emerged as an important regulator of medulla growth and Aire⁺ mTEC differentiation (27). However, it remains unknown whether alterations in the RANK/RANKL axis during aging could contribute to involution and whether RANKL might act as a rejuvenating cytokine for thymic function.

Here, we show that RANKL expression decreases during aging in RANKL-producing thymic cells and that in vivo RANKL neutralization mimics age-related involution, revealing that impaired RANK/RANKL axis contributes to thymic involution. We generated a novel transgenic mouse model lacking RANK specifically in endothelial cells (EC) and provide the first evidence that in addition to TEC, thymic EC are dependent on RANK signaling for their cellularity and functional maturation and are thus highly affected by age-dependent RANKL deprivation. Importantly, ex vivo RANKL treatment of aged mice restores EC maturation and TEC cellularity which increases T-cell progenitor recruitment to the thymus and T-cell production. RANKL-induced thymic rejuvenation leads to peripheral T-cell renewal, enhancing anti-tumor and vaccine responses. Furthermore, we show that RANK and RANKL expression is conserved in human thymic cells and that RANKL stimulation increases EC and TEC cellularity in human thymic organo-cultures. Overall, we identify that alterations in the RANK/RANKL axis contribute to thymic involution and that RANKL cytokine-based treatment is a potent approach to rejuvenate thymic function and improve T-cell immunity in the elderly.

### Materials & Methods

### Mice

CD45.2 and congenic CD45.1 WT (Janvier Labs), Rag2^{-/-} (69) OT-II (70), Ciita^{III+IV-/-} (71) and K14xCiita^{III+IV-/-} (34), Cdh5^{CRE-ERT2} (72) and Tnfrsf11a^{flox} (RANK^{flox}) (73) mice were on a C57BL/6 background and maintained under specific pathogen-free conditions. OT-II mice were backcrossed on a Rag2^{-/-} background. Female aged mice (12- and 18-month-old) were obtained from Janvier Labs. All procedures involving animals were performed in compliance with governmental, institutional and ethical guidelines and were approved by the Marseille Ethical committee for Animal experimentation (Comité National de Réflexion Ethique sur l'expérimentation Animale no. 14).

### Human Thymic Samples

Human thymic samples were obtained from donors requiring a cardiac surgery. Pediatric samples are part of a collection authorized by the French Ministry of Research (Hôpital Necker, Paris, DC-2014-2272) and samples from adults (Hôpital Européen Georges Pompidou) received the authorization from CPP8 Ile-de-France (THYMAGE CPP 17 05 29,23 May 2017). Sex and age of donors are listed in Table S1.

### Human Thymic Organ Cultures

2 mm³ pieces of 0 to 6-month-old human thymi were prepared using precision punch tip. Thymic pieces were transferred into 24-well tissue culture plates on cell culture insert (0.4 μm pores, Millicell) in complete medium (10% FCS, 1% penicillin/streptomycin, HEPES, sodium pyruvate in DMEM Glutamax) containing or not 4 μg/ml of soluble human RANKL protein (Peprotech) and cultured for 2 days at 37° C. Thymic samples were then embedded in OCT for immunofluorescence analyses or enzymatically digested with Liberase TM (Roche, 50 μg/ml) and DNase I (Roche, 100 μg/ml) for TEC and EC analysis by flow cytometry.

### RANKL Stimulation and Anti-RANKL Neutralization in In Vivo Experiments

The recombinant mouse RANKL-GST fusion protein was produced as previously described (74). Mice were injected intravenously with RANKL-GST (5 mg/kg) or GST (5 mg/kg) proteins at days 0, 1 and 2. For RANKL neutralization, mice were injected intravenously with 100 μg of low endotoxin and azide free (ultra-LEAF) neutralizing anti-RANKL antibody (IK22/5, BioLegend) or PBS at days 0, 2 and 4.

### Isolation of Mouse and Human Cell Types

Mouse thymic and splenic cells were isolated by scratching the organs on a 70-μm cell strainer. Splenic red blood cells were lysed with RBC lysis buffer (eBioscience). Mouse BM cells from the femur and tibia were flushed and red blood cells were lysed with RBC lysis buffer. Mouse thymic EC and TEC were isolated as previously described (35) by enzymatic digestion with Liberase TM (50 μg/ml) and DNase I (100 μg/ml) (Roche). CD45⁺ hematopoietic cells were depleted using a biotin anti-CD45 antibody and anti-biotin microbeads, and an AutoMACS Pro separator using the depleteS program (Miltenyi Biotec). Human thymic TEC and EC were isolated using a Multi Tissue Dissociation Kit and a gentle MACS dissociator (Miltenyi Biotec). CD45⁺ hematopoietic cells were depleted using anti-human CD45 microbeads and an AutoMACS Pro separator using the depleteS program (Miltenyi Biotec). The other thymic cell subsets were isolated by scratching the human thymic biopsies on a 70-μm cell strainer.

### Flow Cytometry

Sample preparation and staining were performed using standard procedures. Mouse and human antibodies and reagents used are listed in Table S2. CCR7 staining was performed for 45 min at 37° C. For RANKL detection, cells were incubated for 3 h with Brefeldin A (5 μg/mL; Biosciences) before staining. For intracellular staining of RANKL, Aire, Fezf2, Foxp3 and active caspase 3, cells were fixed, permeabilized and stained using the transcription factor staining kit according to the manufacturer's instructions (Invitrogen). For intracellular staining of IFNγ and Gzmb, cells were stimulated for 5 h at 37° C. with phorbol 12-myristate 13-acetate (PMA; 10 ng/ml; Sigma) and ionomycine (1 μg/mL; Sigma) in the presence of Brefeldin A (5 μg/mL; BD). Cells were fixed, permeabilized and stained using the Cytofix/Cytoperm kit according to the manufacturer's instructions (BD Bioscience). Flow cytometry data were acquired using an LSR II, an LSR Fortessa (BD Bioscience) or a Cytek Aurora (Cytek) and analyzed using FlowJo software (BD Life sciences). EC samples were concatenated and analyzed using the t-SNE plugin of the FlowJo software for unbiased clustering. The phenograph plugin was used for the unsupervised discrimination of EC subsets, which were visualized on the t-SNE dimensional reduction and quantified.

### RT-qPCR

Total RNA was isolated with TRIzol (ThermoFisher Scientific) and cDNA was synthesized with random oligo-dT primers and Superscript II reverse transcriptase (ThermoFisher Scientific). qPCR was performed with ONEGreen Fast qPCR premix (Ozyme) on an ABI 7500 fast real-time PCR system (Applied Biosystem). Reactions were conducted in duplicates. Results were normalized to actin mRNA.

### Fetal Thymus Organ Cultures

C57BL/6 embryonic day 15.5 thymic lobes were cultured for 5 days at 37° C. on polycarbonate Transwell insert (0.4 μm pores, Corning) in a complete medium supplemented with 1.35 mM 2-deoxyguanosine (2dGUO, Sigma) to deplete hematopoietic cells. Lobes were then extensively washed in complete medium and stimulated for 4 days with 2 μg/ml of RANKL-GST or GST control proteins. Thymic lobes were then enzymatically digested with Liberase TM (50 μg/ml) and DNase I (100 μg/ml). Cells were then stained and analyzed by flow cytometry.

### Melanoma Model

Mice received 0.3×10⁶ B16F10 melanoma cells intradermally into both flanks 21 days after GST or RANKL treatment. Tumor volumes were measured using a digital caliper every 2 days starting at day 10 after B16F10 injection and were calculated as (length×width²)/2. For ICBT, 10 mg/kg of anti-PD1 (clone RMP1-14), anti-CTLA-4 (clone 9H10) or isotype control antibodies (clones RTK2758 and SHG-1, respectively) (all from Biolegend) were administered i.p. at days 7, 10, 13 and 16. At day 21, tumors were mechanically and enzymatically digested using Liberase TM (50 μg/ml) and DNase I (100 μg/ml) and filtered through a 70-μm Cell Strainer.

### Immunization

Mice were immunized subcutaneously with 40 μg of OVA₃₂₃₋₃₃₉ peptide in Complete Freund Adjuvant (CFA) in both flanks. 14 days post-immunization, draining LN were analyzed for OVA-specific CD4⁺ T cells using I-A(b)/OVA₃₂₉₋₃₃₇ tetramer (NIH tetramer core facility). Serum was also collected to measure anti-OVA IgG1 titers by sandwich enzyme-linked immunosorbent assay (ELISA) following manufacturer's instructions (Cayman Chemicals).

### Immunofluorescence Staining

20-μm-thick frozen thymic sections were fixed with 2% paraformaldehyde (Merck) and stained with primary and secondary antibodies as previously described(75). Sections were counterstained with DAPI (1 μg/mL; Biolegend) and mounted with Mowiol (Calbiochem). Confocal microscopy was performed with an LSM 880 confocal microscope (Carl Zeiss Microscopy).

### Confocal Image Automated Analysis

Custom Matlab scripts were developed to quantify immunostainings. To evaluate the proportion of keratin-free zones in thymic sections from either young, GST- or RANKL-treated aged mice, images contained 3 channels: cortex-specific K8, medulla-specific K14 and DAPI staining. A complete image section was obtained by averaging the 3 channels. Keratin-free zones were defined as tissue with DAPI⁺ cells but unlabeled for both K8 and K14. Images from each channel were first segmented using the automatic opthr or Otsu method. For the medulla, the automatic threshold was multiplied by 2.2. The resulting binary images were smoothed over 13 pixels (with 0.07-μm pixel size). Complete images were further eroded by 130 pixels to provide a mask to remove staining from the subcapsule. Objects below 4 μm² were discarded. Areas were computed as the number of pixels above threshold for each image. The keratin-free ratio was defined as the keratin-free area divided by the total area. To quantify the number of Aire⁺ cells in medullary regions (K14⁺) from TOC treated or not with RANKL, images from each channel were median filtered over 3 pixels (with 0.12-μm pixel size), segmented using the opthr or moment method, and smoothed over 16 pixels. The automatic threshold was multiplied by 2.5 for Aire and 0.8 for K14. Medulla was defined as K14⁺ areas, dilated by 8 pixels. Density was computed as the number of Aire⁺ cells divided by the surface of the medulla.

### Short-Term Homing Assays

BM cells were obtained from 6-to 10-week-old CD45.1 C57BL/6 mice and red blood cells were lysed. 35×10⁶ cells were injected i.v into CD45.2 recipients. 24 h later, thymi were harvested and cells were stained with a biotinylated anti-CD8α antibody and anti-biotin microbeads. CD8⁺ cells were depleted with an AutoMACS Pro separator using the deplete program (Miltenyi Biotec). CD8⁺-depleted thymocytes were then analyzed for ETP identification by flow cytometry.

### Long-Term Thymopoiesis Assays

BM cells were obtained from 6- to 10-week-old CD45.1/2 C57BL/6 mice and red blood cells were lysed. Cells were stained with a biotinylated anti-CD3ε antibody and anti-biotin microbeads. CD3⁺ cells were depleted on an AutoMACS Pro separator using the depleteS program (Miltenyi Biotec). 35×10⁶ T-cell depleted BM cells were injected i.v into CD45.2 recipients. Four weeks later, thymi and spleen were harvested, stained and analyzed by flow cytometry.

scRNA-seq Experiments

CD31⁺ EC were sorted 3 weeks after GST and RANKL treatment in aged mice (Fig. S6f) and mixed in equal ratios. 500 WT splenic B cells were added to each pool to ease batch correction if necessary. Cells were individually loaded on a chromium Next GEM chip K (10× Genomics) and cells were droplet-encapsulated with a Chromium Controller (10× Genomics). Libraries were prepared with Chromium Single Cell 5′ v2 kit (10× Genomics) according to manufacturer's instructions with dual indexes. The concentration and quality of cDNA libraries were determined using the Qubit dsDNA HS Assay Kit (Thermo Fisher Scientific) for the Qbit fluorometer and the High Sensitivity DNA Analysis Kit (Agilent) for bioanalyzer respectively. Libraries were sequenced on an Illumina NextSeq2000 platform with a 100 cycles P2 flow cell.

scRNA-seq Data Analysis

Raw fastq files were processed using the Cell Ranger software (v6.0.1) from 10× Genomics. Alignment was done on the GRCm38 reference genome. All clustering analyses were performed using the Seurat v4.3.0 package in R v4.2.2 (76). The GST and RANKL scRNA-seq count matrices generated by Cell Ranger were imported into Seurat. For each sample, cells with fewer than 200 genes and genes expressed in fewer than 3 cells were filtered out. Low-quality cells were further removed according to the following thresholds: ˜200-4000 genes detected per cell, ˜0-15000 molecules detected per cell and a mitochondrial count ratio of less than 10%. Doublets were removed using the function ‘doubletFinder_v3’ from the DoubletFinder R package (77). After quality control, 4658 and 4591 cells were kept belonging to the GST- and RANKL-treated samples respectively. Samples were merged using the Seurat function ‘merge’ and the data was log-normalized using the Seurat function ‘LogNormalize’ with ‘scale.factor=10000’. Cells did not separate by cell-cycle phase or experimental condition, as such we did not regress for cell-cycle bias or perform batch correction before our clustering analysis. The top 2000 highly variable genes were found using the variance-stabilizing transform (VST) method and used as input for batch correction. The data was mean centered and scaled, followed by a PCA analysis using the previously identified variable features. The top 15 principal components (PCs) were kept for UMAP embedding and subsequent shared nearest neighbor graph construction. Clusters were found using the Seurat function ‘FindClusters’ set to the default Louvain algorithm with ‘resolution=0.2’. The cluster exhibiting a high expression of the EC marker gene Pecam1 was subsetted and reclustered by rerunning the following: highly variable gene detection, mean centering and scaling, PCA analysis, UMAP embedding and shared nearest neighbor graph construction using the top 12 PCs. Cell clustering was done using the same procedure as above with ‘resolution=0.5’. Marker genes identified by Bautista et al. and Kalucka et al. were used for the classification of capillary, arterial, venous, and choroid plexus like clusters respectively (21, 37). The ‘dittoHeatmap’ function available in the dittoSeq R package (v1.10.0) was used for heatmap visualization(78). For all heatmaps, the average gene expression per cluster was calculated and scaled between 0-1. Dot-plots of key marker genes were generated using the function ‘DotPlot_scCustom’ available in the scCustomize R package (v1.1.1). For EC Gene Set Enrichment Analysis (GSEA), the Seurat function ‘AddModuleScore’ was used to compute per cell module scores of RANK-deficient EC (38) and angiogenesis (39) gene set signatures. Violin-plots were used to visualize the module score distribution in the Venous 1 and the Venous 2 EC clusters. A Wilcoxon rank-sum test with Benjamini-Hochberg (BH) correction was used to assess the statistical significance between the module scores computed for the GST- and RANKL-treated samples. The Nebulosa package was used to visualize features on UMAP embeddings using kernel density estimation (79).

### Data and Code Availability

scRNA-seq data is available on GEO under the accession number GSE241880. Scripts used to analyze scRNA-seq data are available on GitHub (github.com/JessicaChevallier/MIlab_EC_scRNA_thymus).

### Bone Structure Analysis by Micro-Computed Tomography and Histomorphometry

Young (8-week-old), GST- and RANKL-treated aged (12-month-old) mice were injected i.p. with tetracyclin (20 mg/kg; Sigma-Aldrich) and calcein (10 mg/kg; Sigma-Aldrich), 4 and 1 days before bone collection for young mice and 8 and 3 days before bone collection for aged mice. To evaluate if RANKL potential effects on bone architecture could be prevented, mice were pre-treated s.c. or not with risedronate sodium (10 μg/kg; Sigma-Aldrich) 1 day before RANKL treatment and was then co-injected with RANKL. Mice were euthanized 21 days after the first RANKL injection and femurs were collected and fixed in 70% ethanol without decalcification. They were analyzed with high-resolution micro computed tomography (micro-CT) using a Skyscan 1272 microCT (Bruker, Kontich, Belgium). Bones were placed vertically and scanned with the following settings: aluminum filter, 0.5 mm; resolution, 6 μm; energy, 900 kV; intensity, 100 μA; and exposure time, 750 ms. Images were obtained using and analyzed with NRecon, DataViewer, CTAn and CTVox softwares were used for 3D images reconstruction and analyses. Measurements were performed on the distal metaphysis for the trabecular bone and on the diaphysis for the cortical bone. The following morphometric parameters were measured: Bone Volume/Tissue volume (BV/TV, %), Trabecular Thickness (Tb.Th, μm) Trabecular number (Tb.N, 1/μm) Trabecular Separation (Tb.Sp, μm), cortical thickness (Ct.Th, μm).

The femur from each animal was collected after microCT measurement, dehydrated in xylene and then embedded without demineralization in methylmethacrylate. 5-μm-thick coronal sections were cut parallel to the long axis of the femur, using an SM2500S microtome (Leica, Germany). Sections were stained with toluidine blue to quantify osteoblastic and osteoid surfaces (Ob.S/BS, OS.S/BS respectively). Serial 5-μm-thick sections were stained with naphthyl phosphate (Sigma-Aldrich) and counterstained with aniline blue to evaluate the bone resorption through tartrate-resistant acid phosphatase (TRAP), expressed as osteoclast surfaces (Oc.S/BS) and osteoclast number (Nb.OC/mm²). To evaluate bone formation rate, the double fluorescent labeling of tetracycline and calcein was analyzed. Two 11-μm-thick unstained sections were taken for measurement of the dynamic parameters under UV light. The matrix apposition rate (MAR) was measured using the Microvision image analyzer (Evry, France) by a semiautomatic method using tetracycline and calcein double-labeled bone surfaces. The mineralizing surfaces (MS/BS) were measured in the same areas using the objective eyepiece Leitz integrate plate II and the bone formation rate (BFR) was calculated using the two measured parameters. All histomorphometric parameters were recorded in compliance with the recommendations of the American Society for Bone and Mineral Research Histomorphometry Nomenclature Committee(80).

### Statistical Analysis

Statistical significance was assessed with GraphPad Prism v9.3.1 software using Mann-Whitney test, two-way ANOVA or Wilcoxon rank-sum test with Benjamini-Hochberg correction. *, p<0.05; **, p<0.01; ***, p<0.001, ****, p<0.0001. Error bars represent mean±SD or mean±SEM for tumor growth.

### Results

### Reduced RANKL Expression in the Thymus During Aging

Since RANK signaling controls mTEC cellularity and differentiation(27), both of which are impaired during aging, we hypothesize that alterations in the RANK/RANKL axis may contribute to thymic involution. We first monitored by flow cytometry the expression of the receptor RANK in TEC subsets of 8-week-, 6- and 12-month-old wild-type (WT) mice. Total TEC, cTEC and mTEC numbers decreased during aging (data not shown). Both mTEC^{lo} and mTEC^{hi} also decreased with an mTEC^{hi}/mTEC^{lo} ratio that progressively decreased during aging. Despite a reduced TEC cellularity, RANK levels remained relatively stable in cTEC and mTEC during aging. We reanalyzed single-cell RNA-seq (scRNA-seq) datasets (28, 29) and found that most mTEC subsets as well as some cTEC express Tnfrsf11a, the gene encoding RANK. Unexpectedly, thymic CD31⁺ EC were also reduced and expressed substantial levels of RANK in both young and aged mice. We confirmed by histology that EC expressed RANK. Interestingly, RANK expression in thymic EC scRNA-seq dataset (30) was mainly detected in cells belonging to the venous Selp⁺ EC cluster, implicated in T-cell progenitor homing.

We then measured RANKL expression during aging in thymic hematopoietic cells. The cellularity of all thymocyte subsets from the early T-cell progenitor (ETP) to the SP stage, including innate lymphoid cells, strongly decreased from 6 months of age while their frequencies remained relatively stable, indicating the maintenance of a sufficient number of RANKL-producing cells in proportion to thymic size (data not shown). Whereas the expression of RANKL in conventional CD4⁺ T cells (CD4⁺ Tconv) and natural killer T (NKT) cells slightly decreased with age, a substantial decrease was observed in lymphoid tissue inducer (LTi) cells and γδ T cells. Interestingly, γδ T cells, that expressed the highest RANKL level were in close contact with EC, similarly to CD4⁺ SP thymocytes. Since not only emigrating mature T cells, but also immigrating ETP interact with RANK-expressing EC and TEC, we also assessed RANKL expression in ETP and found that they expressed substantial levels of RANKL, which decreased during aging. Altogether, our data reveal that decreased RANKL expression in thymic-producing cells could affect RANK-expressing TEC and EC during aging.

### In Vivo Neutralization of RANKL in Young Mice Mimics Thymic Involution

To test the hypothesis that decreased RANKL availability during aging may be involved in thymic involution, we treated young 8-week-old WT mice with a neutralizing anti-RANKL antibody and analyzed their thymi one week after the first injection (data not shown). Untreated young and 6-month-old WT mice were used as controls. Interestingly, similarly to 6-month-old thymi, RANKL neutralization in young mice induced the appearance of keratin-free zones devoid of TEC, which is a hallmark of thymic aging (2, 3). Remarkably, RANKL neutralization resulted in a reduced thymic cellularity affecting thymocytes from the DN1 to the SP stage. Consistent with mTEC dependency on RANK signaling (27), the mTEC/cTEC ratio and mTEC numbers were drastically reduced. Since EC expressed RANK, we hypothesized that these cells could also be affected by in vivo RANKL neutralization. Similar to 6-month-old mice, RANKL neutralization in young mice resulted in a strong reduction in EC numbers, including thymic portal EC (TPEC; P-sel⁺Ly6c⁻), implicated in T-cell progenitor homing (31).

We next investigated whether RANKL neutralization affects the survival of TEC and EC by quantifying apoptotic cells via the detection of active caspase 3. An increased proportion of apoptotic cells was observed in cTEC, mTEC^{lo} and mTEC^{hi} of 6-month-old mice compared to 8-week-old young mice, suggesting an increased apoptosis of these cells during aging (data not shown). Remarkably, RANKL neutralization in young mice resulted in a 2-fold increase of mTEC^{hi} apoptotic cells in contrast to mTEC^{lo} and cTEC whose survival does not appear to depend on RANK signaling. As opposed to TEC, only a small proportion of apoptotic cells, which did not vary during aging or upon RANKL neutralization, was observed in thymic EC.

We then analyzed the impact of RANKL neutralization on mTEC and EC functional maturation. As in 6-month-old thymi, this treatment drastically reduced Aire⁻Fezf2⁺ and Aire⁺Fezf2⁺ mTEC cellularity (data not shown). Furthermore, as observed in 6-month-old mice, RANKL neutralization resulted in a reduced expression of the EC adhesion molecules ICAM-1 and VCAM-1, implicated in T-cell progenitor homing (32, 33). The expression of these markers in EC subsets, including CD31 and Ly6C, was then used as input for t-distributed stochastic neighbor embedding (t-SNE) dimensionality reduction and PhenoGraph clustering for unsupervised EC cluster identification in anti-RANKL-treated or untreated young mice. This analysis identified fourteen clusters that were classified according to their relative expression of functional markers as immature (clusters 1 to 5), semi-mature (clusters 6 to 9) and mature/functional (clusters 10 to 14). Cluster quantification revealed an enrichment in immature (2, 4, 5) and semi-mature (8, 9) subsets upon RANKL neutralization, while two of the most mature/functional subsets (11 and 13) drastically declined. Importantly, cell numbers of mature/functional clusters were strongly diminished. In accordance with the altered number and functional phenotype of EC, ETP numbers were reduced in anti-RANKL-treated mice compared to controls. Our results reveal that the RANK-RANKL axis is crucial for the functional maturation of thymic EC and T-cell progenitor recruitment. Overall, our data show that RANKL neutralization in young mice mimics involution by inducing artificial aging of the thymus and that RANKL decrease observed during aging may, therefore, contribute to thymic involution.

### RANKL-Expressing CD4⁺ SP Thymocytes Promote RANK-Dependent EC Functional Maturation

Since EC expressed RANK and were strongly affected by RANKL neutralization, we sought to identify the cellular source(s) of RANKL involved in EC maturation. We first evaluated the respective contribution of CD4⁺ SP thymocytes that are absent in mice deficient for the promotor III and IV of the class II transactivator (Ciita^{-/-}) and restored by introducing a Ciita-transgene driven by a cTEC specific promoter (Ciita-Tg) (34, 35) (data not shown). Interestingly, TPEC frequencies and numbers were substantially reduced in Ciita^{-/-} mice and fully restored in Ciita-Tg mice. Furthermore, the expression of ICAM-1 and P-sel was reduced in Ciita^{-/-} mice and restored in Ciita-Tg mice, showing that CD4⁺ SP thymocytes participate in EC maturation. Interestingly, a substantial proportion of thymic EC expressed MHCII molecules, which were completely abrogated in Ciita^{-/-} mice. Even though MHCII expression was only weakly restored in Ciita-Tg mice, these mice had normal EC number and phenotype, suggesting that MHCII expression is dispensable for EC maturation.

We then analyzed EC number and phenotype in Rag2^{-/-} mice that lack the main RANKL-producing thymic cells. A profound defect in EC numbers and maturation was observed in these mice. To evaluate whether the reintroduction of CD4⁺ SP thymocytes was sufficient to restore EC number and phenotype, we took advantage of Rag2^{-/-} OT-II transgenic mice that possess only CD4⁺ SP thymocytes expressing an ovalbumin-specific TCR. Importantly, OT-II CD4⁺ SP thymocytes expressed a normal level of RANKL. Compared to Rag2^{-/-} mice, these mice displayed a normal number of EC despite a slight restoration of TPEC and a partial restoration of ICAM-1 and VCAM-1 expression levels, further demonstrating that CD4⁺ SP thymocytes contribute to EC functional maturation. Moreover, the administration of the recombinant RANKL-GST protein (hereafter referred as RANKL) in Rag2^{-/-} mice partially restored EC and TPEC numbers and increased the expression of adhesion molecules compared to GST-treated Rag2^{-/-} controls, demonstrating that RANKL stimulates EC maturation. To confirm that RANKL directly acts on EC, we stimulated with RANKL or the GST control protein fetal thymic organ cultures (FTOC) that only contain stromal cells upon 2dGUO treatment that deplete hematopoietic cells. Strikingly, EC from RANKL-treated FTOC showed an upregulation of ICAM-1, P-sel and CD31 and increased frequencies of P-sel⁺ICAM-1⁺ mature EC compared to GST-treated controls. To prove that RANK in EC controls their cellularity and functional maturation, we generated a novel transgenic mouse model in which RANK was conditionally deleted on EC using Cdh5^{CRE-ERT2}×Tnfrsf11a^{fl/fl} mice (called EC^{ΔRANK} mice). We first confirmed that Tnfrsf11a (RANK) expression by thymic EC was abrogated upon tamoxifen treatment without altering Tnfrsf11a expression in TEC and their representation. Importantly, the number of EC and their functional phenotype were altered in EC^{ΔRANK} mice compared to their littermate controls, confirming that RANK signaling in EC controls their functional maturation. Strikingly, thymic cellularity was strongly reduced, affecting T-cell subsets from the DN to the CD4⁺ and CD8⁺ SP stages. These data firmly demonstrate that thymic EC maturation depends on RANK signaling and partial RANKL deficiency is sufficient to alter EC number and maturation.

### RANKL Treatment Rejuvenates Thymic Function in Aged Mice

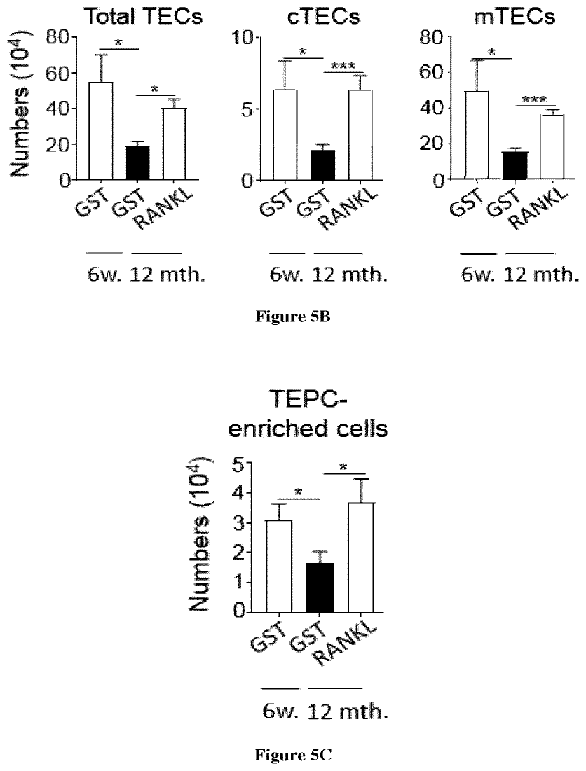

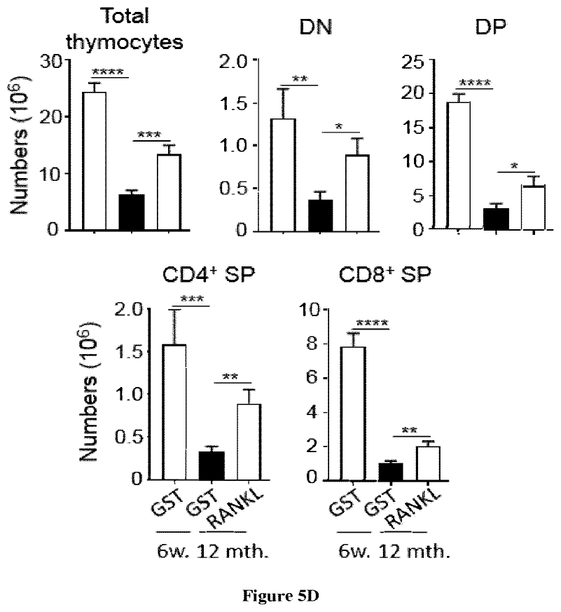

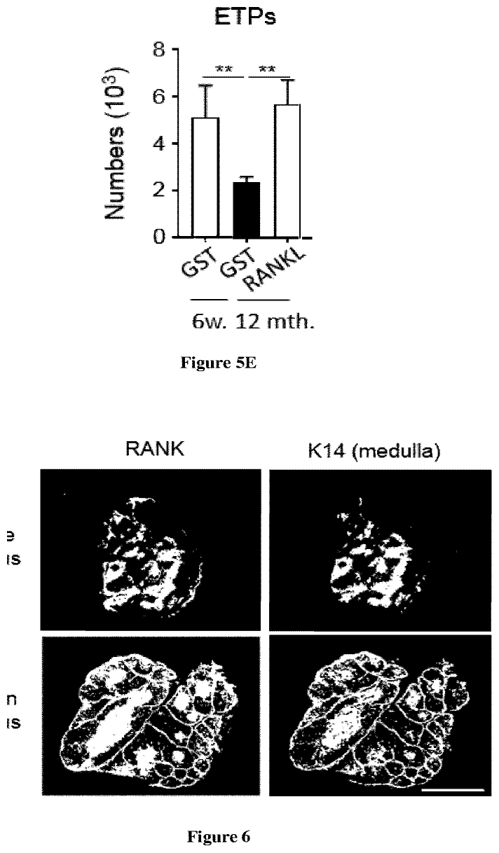

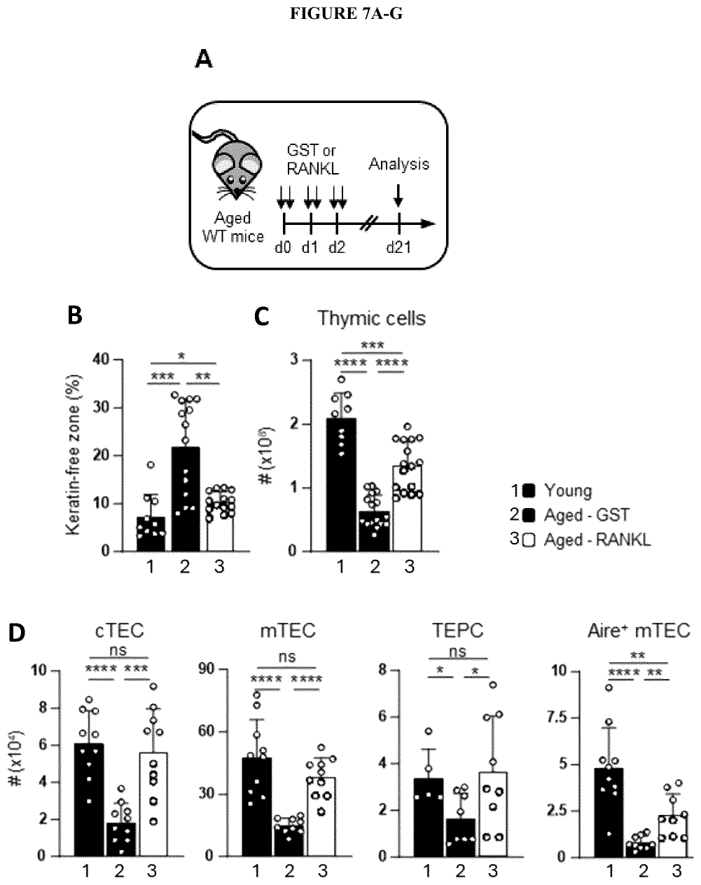

Since RANKL expression decreases with age while RANK levels on EC and TEC remain comparable to young mice, we hypothesized that RANKL treatment in aged mice could reverse thymic involution. We set-up a short-term 3-day treatment with either RANKL or GST proteins in 12-month-old mice and analyzed the thymus three weeks later, corresponding to thymopoiesis duration in mice (FIG. 7A). Young 8-week-old mice were used as controls. We first evaluated thymic architecture after RANKL treatment by histology. While the thymus of aged mice was smaller than that of young mice, RANKL treatment remarkably increased thymus size. Moreover, similar to RANKL neutralization in young mice, aged thymi showed numerous TEC- and EC-free zones, which were almost absent in young and RANKL-treated aged mice (FIG. 7B). These results reveal that RANKL treatment restored both thymic size and architecture in aged mice.

Accordingly, thymic cellularity increased in RANKL-treated aged mice compared to GST-treated aged controls (FIG. 7C). Remarkably, both cTEC and mTEC numbers were restored to levels close to those of young mice (FIG. 7D). This restoration was observed from thymic epithelial progenitor enriched cells (TEPC) to the mTEC^{hi} stage, including Aire⁺ mTEC^{hi} (FIG. 7D). Of note, RANKL-induced thymic rejuvenation was also efficient in 18-month-old mice, discussed above. Similar to 6-month-old mice, 12-month-old mice showed an increased proportion of apoptotic TEC subsets (FIG. 7E,F), confirming that TEC apoptosis strongly increases with age. In contrast to cTEC and mTEC^{lo}, RANKL-treatment reduced the proportion of apoptotic mTEC^{hi} compared to GST-treated aged controls, further demonstrating that RANKL stimulation promotes the survival of mTEC^{hi} (FIG. 7E,F). In line with TEC regeneration, thymocyte numbers from the DN1 to the CD8⁺ and CD4⁺ SP stages, including Foxp3⁺CD25⁺ regulatory T cells (Treg) and their precursors, substantially increased in RANKL-treated aged mice (FIG. 7G). In contrast, their frequencies remained unaltered, indicating that RANKL quantitatively promotes T-cell production without causing a disequilibrium among T-cell subsets. Altogether, these results reveal that RANKL treatment rejuvenates TEC subsets, thereby boosting T-cell production in aged mice.

### RANKL Restores Thymic EC Functional Maturation and T-Cell Progenitor Homing in Aged Mice

Numbers of total EC, including TPEC, were substantially reduced in 12-month-old mice compared to young 8-week-old mice (data not shown). Their maturation was severely altered, as evidenced by the imbalance between immature and mature/functional EC subsets using an unsupervised flow cytometry analysis. Strikingly, EC numbers, including TPEC, as well as their maturation were efficiently restored upon RANKL treatment. To analyze in depth the impact of RANKL treatment on thymic EC, we performed a scRNA-seq on total CD31⁺ EC purified from GST- and RANKL-treated aged mice. Using known EC markers (21, 36), our analysis revealed six EC clusters: two capillary, two venous, one arterial and one that resembles recently described choroid plexus EC (37). Given that venous cells are responsible for T-cell progenitor homing (31) and expressed RANK, we focused our analysis on the two venous clusters. We computed module scores to assess the expression of RANK-deficient EC (38) and angiogenesis (39) signatures and observed that venous EC of RANKL-treated mice not only upregulated a RANK/RANKL response but also an angiogenic signature compared to GST-treated aged mice. In line with our unsupervised flow cytometry analysis, the adhesion molecules Selp, Sele and Vcam1 were upregulated upon RANKL treatment. Interestingly, Ltbr, implicated in EC differentiation (31), Dll1 and Kitl, critical for ETP development (40, 41), were also upregulated. Moreover, Bst1, associated with T-cell egress from the thymus (30), was upregulated; while Cldn5, a tight junction protein was downregulated, which likely facilitates thymic egress (42). Interestingly, a high-endothelial venules (HEV)-like signature was upregulated in the venous 2 cluster (43), including the upregulation of Glycam1 and Cd34 that interact with CD62L expressed by ETP, facilitating their recruitment to the thymus (44, 45).

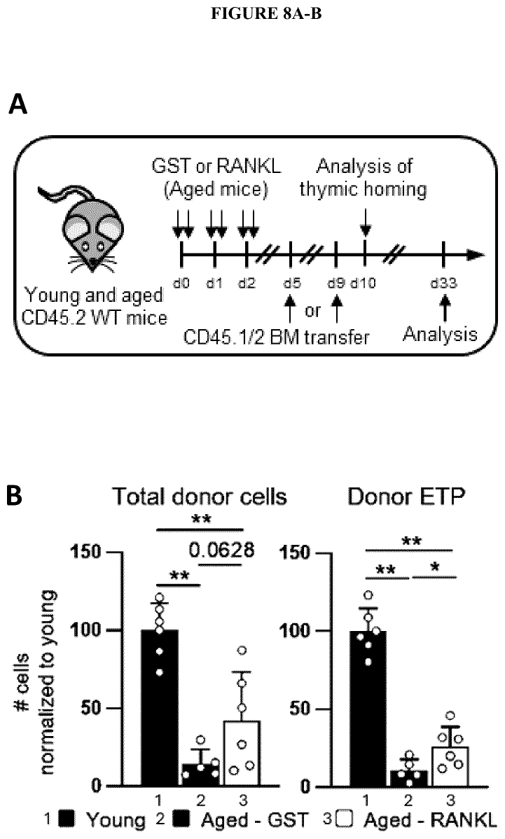

In line with the regeneration of functional EC, the number of ETP, which strongly reduced with age, was fully restored in RANKL-treated aged mice (data not shown). Notably and as previously suggested (46), RANKL treatment promoted the mobilization of common lymphoid progenitors from the bone marrow (BM) while the proportion of their BM lymphoid progenitors was unaffected. Furthermore, B-cell development in the BM was unchanged by RANKL treatment. To define whether the increase in ETP observed after RANKL treatment was due to an improved recruitment of T-cell progenitors, short-term homing assays were performed by injecting the same number of T-cell depleted CD45.1 congenic BM cells into unmanipulated RANKL- or GST-treated aged mice and young controls (FIG. 8A). Strikingly, the receptive capacity of circulating progenitors was strongly reduced in aged mice compared to young mice (FIG. 8B). Interestingly, the thymus of RANKL-treated aged mice imported two times more ETP than GST controls, demonstrating that RANKL treatment improves thymic capacity to recruit T-cell progenitors in the aged thymus.

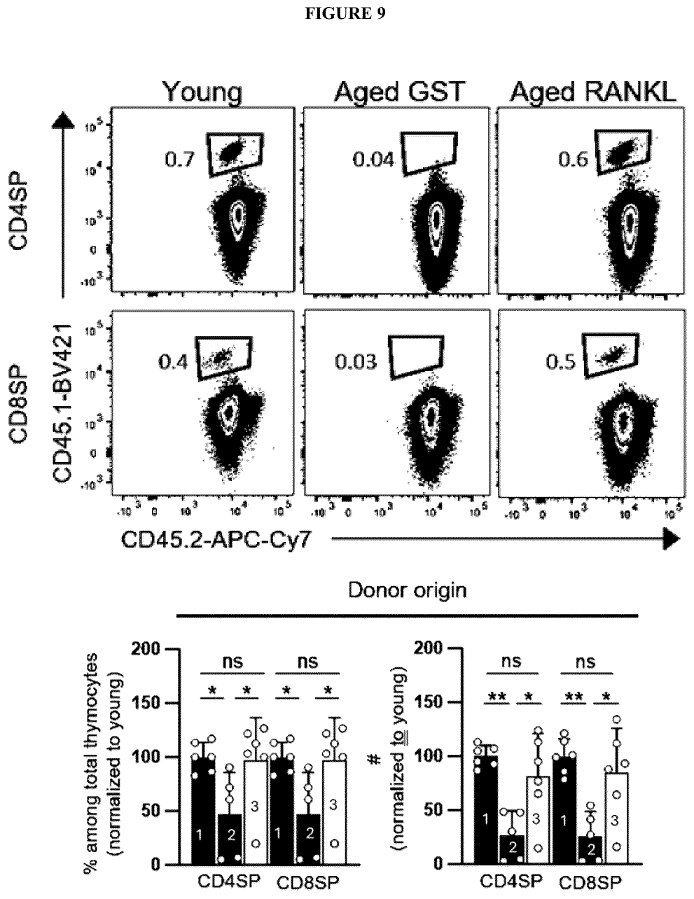

To evaluate to which extent RANKL-induced thymic rejuvenation in aged mice increases T-cell production from the ETP stage, we used the same experimental setup, but injected T-cell depleted CD45.1/2 congenic BM cells and analyzed T-cell development of donor origin four weeks later (FIG. 8A). As expected, GST-treated aged controls produced fewer CD4⁺SP and CD8⁺SP thymocytes of CD45.1/2 donor origin compared to their younger counterparts (FIG. 9). In line with TEC and EC regeneration and the increased ETP recruitment, RANKL treatment increased the generation of CD4⁺SP and CD8⁺SP thymocytes by 3-fold, a similar level observed in young mice. Thus, the rejuvenated thymus produces more mature T cells than the aged thymus from the same amount of circulating progenitors over the same time period by acting on the regeneration of functional mature TEC and EC.

### RANKL-Induced Thymic Rejuvenation Renews Peripheral T-Cell Pool Without Major Adverse Effects on Bone in Mice

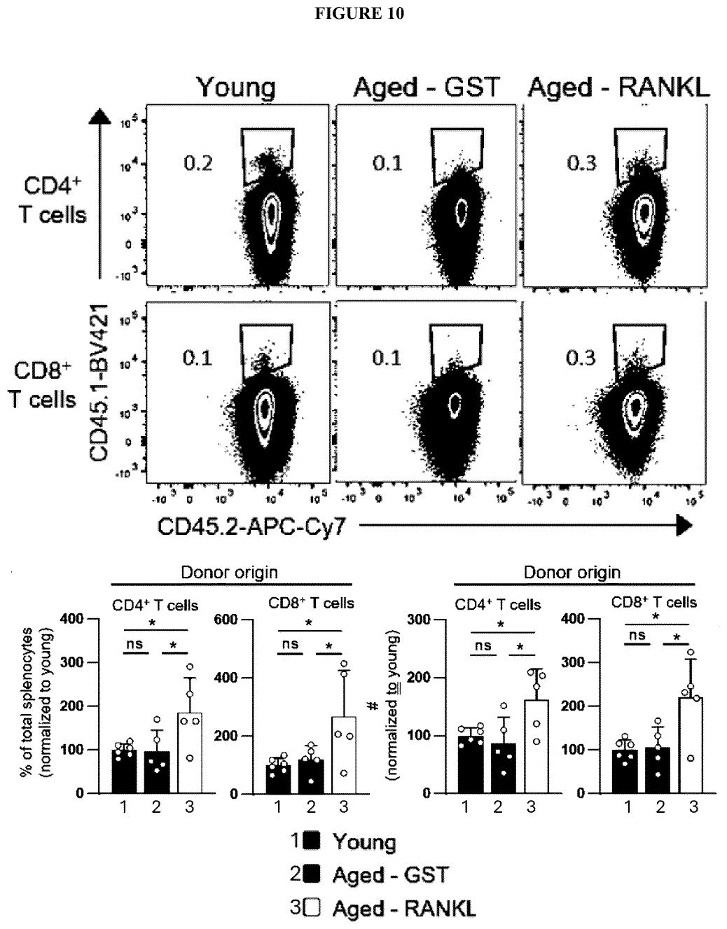

The purpose of thymic regenerative therapies in aged mice is to renew the peripheral T-cell pool to restore effective T-cell immune responses. To assess the renewal capacity of the peripheral T-cell pool upon RANKL-induced thymic rejuvenation, we analyzed one month later the amount of newly produced T cells reaching the periphery in our adoptive transfer setup (FIG. 8A). Compared to GST-treated aged controls, we found a 2-fold increase in the numbers of newly produced CD4⁺ and CD8⁺ T cells of CD45.1/2 donor origin in the spleen of RANKL-treated aged mice (FIG. 10).

Since RANKL is known to promote osteoclast formation and bone resorption(47), we evaluated the safety of RANKL treatment on bone in our thymic rejuvenation setup (FIG. 7A). The anti-bone-resorbing agent, risedronate, was administered or not on the same days as RANKL and one day before for prevention, as previously described (48) (data not shown). In young 8-week-old mice, RANKL increased cortical thickness. At the trabecular bone, RANKL induced a reduction in the bone volume related to an increase in trabecular separation, thickness and osteoclast surface, indicating an enhanced bone resorption in young mice. These changes were prevented by the concomitant administration of RANKL with risedronate. Moreover, RANKL had no effect on bone formation in young mice, as evidenced by a similar bone formation rate to control mice. In contrast, RANKL had no effect on any bone formation parameter in aged mice. Importantly, risedronate administration did not alter RANKL treatment efficacy on thymic rejuvenation in aged mice. Altogether, RANKL-induced thymic rejuvenation has the capacity to renew the peripheral T-cell pool in aged mice without major adverse effects on bone in our thymic rejuvenation setup.

### RANKL-Induced Thymic Rejuvenation Enhances T-Cell Immunity

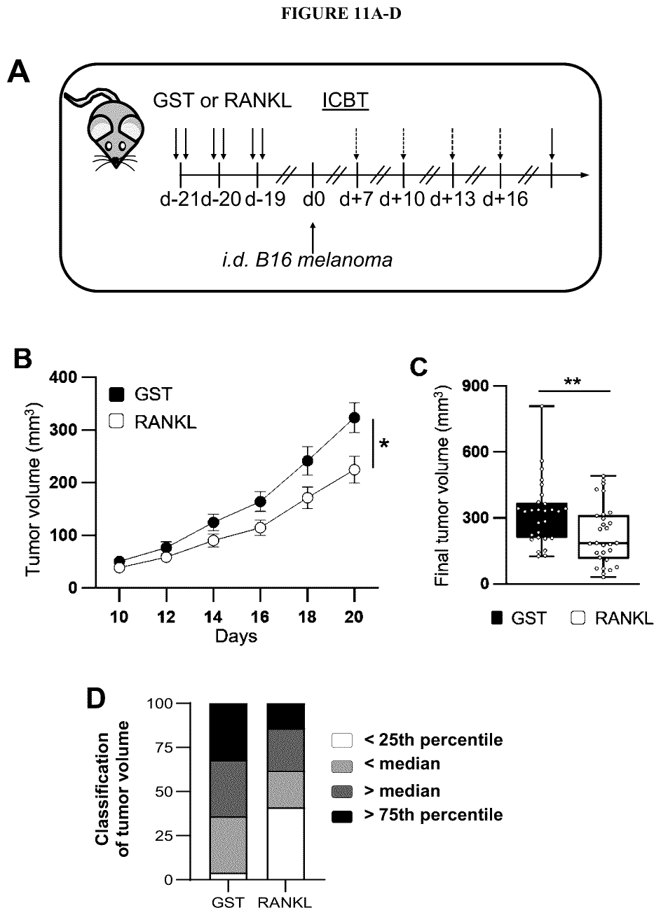

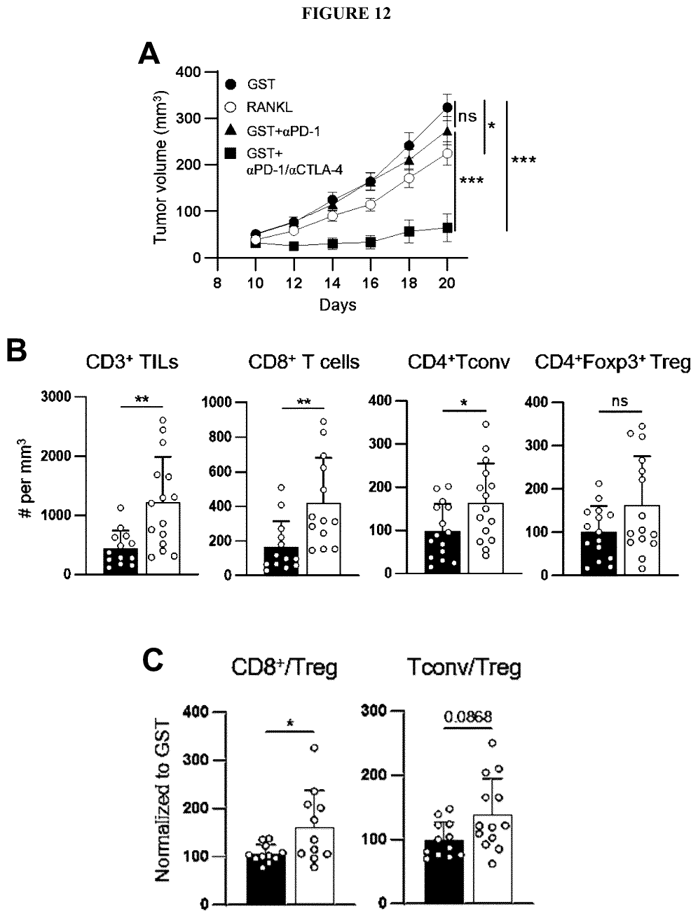

Since RANKL treatment in aged mice efficiently restored thymic T-cell production (FIG. 7), which increased newly produced T cells in the periphery (FIG. 8), we next assessed whether this treatment enhances immune responses. To this end, three weeks after RANKL-induced thymic rejuvenation, aged mice were intradermally injected with syngeneic B16F10 melanoma cells (FIG. 11A). Tumor growth was substantially attenuated in RANKL-treated mice compared to GST-treated controls (FIG. 11B) and final tumor volumes were reduced (FIG. 11C,D). We then compared the efficacy of our treatment to that of immune checkpoint blockade therapy (ICBT). In aged mice, the combination of anti-PD1 and anti-CTLA4 antibodies dramatically inhibited melanoma growth in comparison to anti-PD1 treatment alone (FIG. 12A). Interestingly, compared to GST-treated mice, tumor growth was reduced in RANKL-treated aged mice but not in anti-PD1-treated mice, suggesting that RANKL treatment had a superior effect than anti-PD1 therapy alone. Moreover, the administration of anti-PD1 antibody in RANKL-treated mice had a slight synergistic effect with a final tumor volume that tended to be smaller (data not shown).

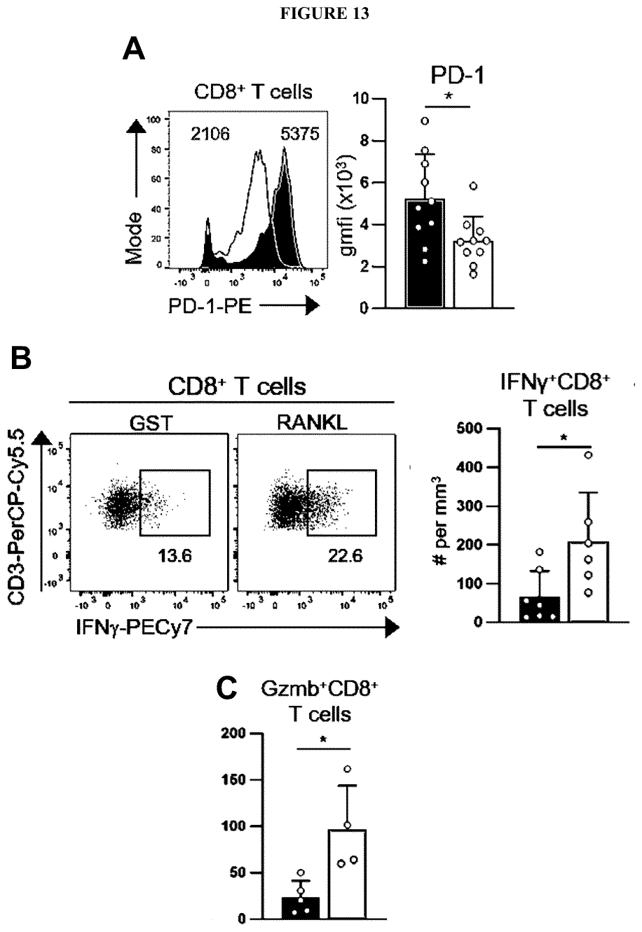

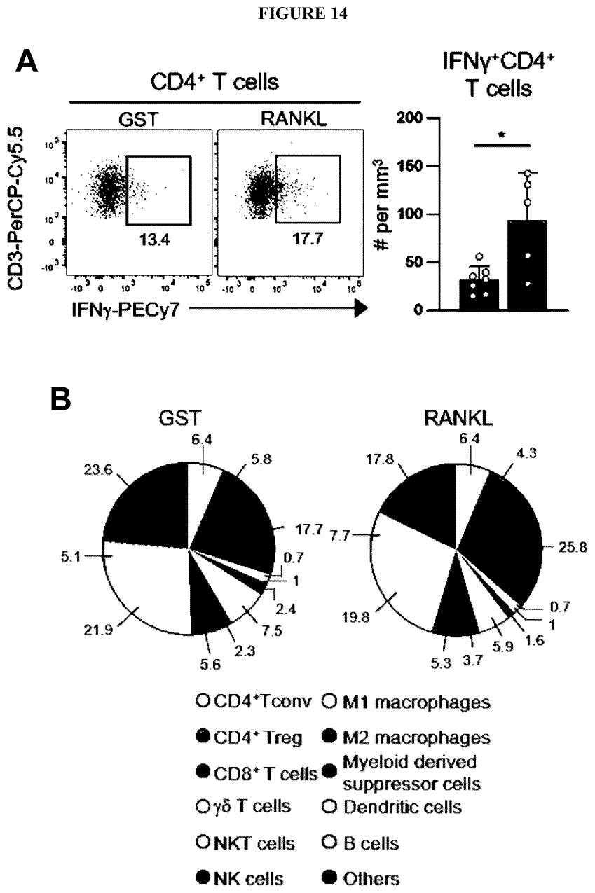

We then found an increased density of CD3⁺ tumor-infiltrating lymphocytes (TILs) in tumors of RANKL-treated mice compared to GST-treated controls (FIG. 12B). This increase reflects a substantial infiltration of CD8⁺ T cells and CD4⁺ Tconv, while CD4⁺Foxp3⁺ Treg remained unchanged, resulting in higher CD8⁺/Treg and CD4⁺ Tconv/Treg ratios (FIG. 12C). CD8⁺ TILs had a reduced PD-1 expression in RANKL-treated mice compared to GST controls (FIG. 13A), indicating that CD8⁺ TILs had a less exhausted phenotype, consistent with peripheral T-cell renewal induced by RANKL-induced thymic rejuvenation. Importantly, tumors from RANKL-treated mice showed increased densities of IFNγ- and granzyme-producing CD8⁺ TILs and IFNγ-producing CD4⁺ TILs (FIG. 13B,C, 14A). Strikingly, we found a higher infiltration mainly of CD8⁺ T cells in tumors from RANKL-treated mice in contrast to other immune cells (FIG. 14B). These findings provide strong evidence that RANKL-induced thymic rejuvenation enhances anti-tumor CD4⁺ and CD8⁺ T-cell immunity in aged mice.

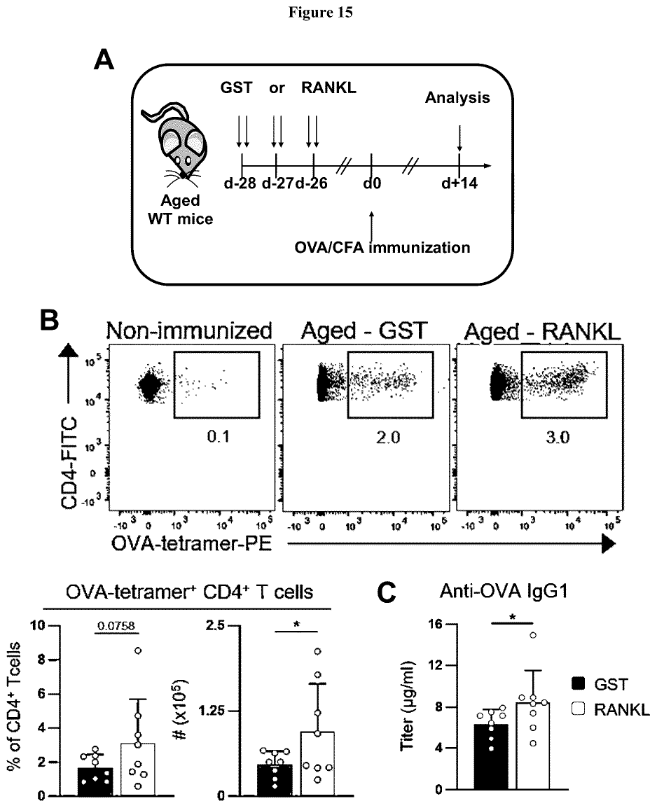

Given that aging results in impaired vaccine responses (49), we assessed whether RANKL-induced thymic rejuvenation improves T-cell responses directed towards a neoantigen. To this end, GST- and RANKL-treated aged mice were immunized upon thymic rejuvenation with an MHCII-restricted ovalbumin peptide (OVA₃₂₃₋₃₃₉) (FIG. 15A). Interestingly, the numbers of OVA-specific CD4⁺ T cells and the titers of OVA-specific IgG1 antibodies increased in RANKL-treated mice compared to GST controls (FIG. 15B,C). Altogether, T-cell renewal induced by thymic rejuvenation improves T-cell mediated anti-tumor immunity and T-cell dependent antigen-specific responses upon immunization.

### RANKL Stimulates Human EC and TEC

From a therapeutic perspective, we evaluated the translational potential of our findings in humans. Frequencies among total thymic cells of CD4⁺SP thymocytes and γδ T cells tended to decrease in infants older than 6 months compared to younger individuals (data not shown). As observed in mice, CD4⁺SP thymocytes, NKT and γδ T cells expressed RANKL. Since several subsets of innate lymphoid cells (ILC) were identified in the human thymus(50), including ILC1, ILC2, ILC3 and LTi-like cells, we assessed RANKL expression levels in these subsets. All of them expressed RANKL, although the levels were higher in LTi cells. Interestingly, similar to mice, RANKL expression was reduced in CD4⁺SP and γδ T cells in older infants (>6 months).

Importantly, human cTEC, mTEC and EC expressed RANK, suggesting that they could respond to RANKL stimulation (data not shown). Because human thymic EC remain poorly characterized, we reanalyzed a scRNA-seq dataset of human stromal cells during aging (21) and focused our analysis on EC. We identified five human EC clusters: one arterial, one capillary, one lymphatic and two venous. Interestingly, RANK (TNFRSF11A) was highly expressed in the venous clusters 2 and 3, which we identified as mature EC subsets in line with the high expression of functional markers VCAM1, SELP, ICAM1 and SELE. We further analyzed EC subset distribution during aging and found that the lymphatic EC cluster, expressing SELP, was specifically present at fetal week 19. The capillary cluster, which expressed an immature phenotype was also highly represented at this fetal stage. Interestingly, the venous cluster 3, containing cells expressing functional adhesion molecules, was highly abundant at post-natal day 10 while its representation was diminished in the young 25-year-old adult. Conversely, the proportion of the capillary cluster and the venous cluster 2, expressing a more immature phenotype, increased in the young adult. These data indicate that adult EC are poorly functional compared to fetal and post-natal EC stages, revealing that the composition of human thymic EC is dynamic with age.

We then analyzed thymic architecture of 2-day-old, 15- and 47-year-old patients. To this end, we used a pan-keratin marker, the mTEC-specific keratin 14, and fibronectin that was reported to stain human EC (21). Interestingly, similar to mice (FIG. 7B), numerous zones devoid of TEC and EC were detected in the thymus of 15- and 47-year-old patients compared to 2-day-old neonates (data not shown). In addition, the density of Aire⁺ cells within medullary regions drastically diminished with age. Finally, we evaluated the capacity of RANKL to stimulate human TEC and EC using thymic organo-cultures (TOC). We found by immunofluorescence that RANKL treatment increased the density of Aire⁺ cells in these TOC. Remarkably, compared to untreated TOC, the addition of RANKL increased cTEC and mTEC cellularity by 2-fold and EC cellularity by 1.5-fold. Altogether, our data show that the RANK/RANKL axis is conserved in human thymic cells and provide the proof-of-concept that RANKL could be a novel therapeutic molecule to rejuvenate the thymus in the elderly.

### Discussion

Although alterations in TEC and T-cell progenitor homing are two major factors of thymic involution, the underlying mechanisms of these natural defects remain poorly understood. It has been described that the transfer of lymphoid progenitors from young mice into aged recipients was unable to restore thymic function, suggesting that defects in stromal cells rather than in hematopoietic cells are responsible for thymic involution (51, 52). These findings highlight the need to target stroma cells to rejuvenate thymic function by identifying age-related defective pathways. Here, we provide the first evidence that a decrease in RANKL production by key thymocyte subsets during aging contributes to functional alterations in stromal cells and, thereby, to thymic involution. The reasons for the age-related decrease in RANKL expression in CD4⁺ SP thymocytes, LTi, ETP and γδ T cells require further investigation. Since TGFβRII stimulation in synergy with TCR activation upregulates RANKL expression in CD4⁺ SP thymocytes (53, 54), a plausible explanation is that altered TGFβRII and/or TCR signaling during aging may be responsible for RANKL decrease. This hypothesis is difficult to test since TGFβRII deficiency in T cells results in an inflammatory syndrome by three weeks of age due to impaired T-cell tolerance. Moreover, RANKL reduction in γδ T cells may be explained by a difference in the representation of their subsets in the aged thymus, as described in the early stages of life (55).

We show that RANKL neutralization in young mice mimics thymic involution, demonstrating that a limited amount of available RANKL alters thymic function. This is characterized by a reduction in thymic cellularity, accompanied by the appearance of keratin-free zones, a feature that we observed in mice and humans during aging. As anticipated (27,56), RANKL neutralization reduced mTEC cellularity with a marked impact on functional Aire⁻Fezf2⁺ and Aire⁺Fezf2⁺ mTEC subsets, due to impaired survival and differentiation. Unexpectedly, RANKL neutralization also reduced EC cellularity with a pronounced effect on mature subsets expressing P-selectin, VCAM-1 and ICAM-1 adhesion molecules. Given that EC are crucial for T-cell progenitor recruitment (32), these findings are consistent with a reduced representation of ETP and thymocyte subsets upon RANKL neutralization. Importantly, the heterogeneity and functional properties of thymic EC throughout life remain poorly studied. Our scRNA-seq analysis revealed the existence of capillary, arterial and venous subsets in the mouse thymus. A venous EC subset expressing high levels of P-selectin, VCAM-1 and ICAM-1 adhesion molecules, likely corresponds to functional TPEC, which guide T-cell progenitor recruitment (31). We observed that this complex heterogeneity is conserved in the human thymus, with a strong representation of TPEC at postnatal day 10, followed by a reduction in 25-year-old adults. The dynamic TPEC representation likely correlates with thymic activity observed during the postnatal and adult stages (57), highlighting the importance of functional EC in controlling T-cell production. We demonstrate that RANKL stimulation acts directly on EC. First, we provide the first evidence that RANK is expressed by thymic EC using three distinct approaches: scRNA-seq, flow cytometry and histology. Second, we show that RANKL stimulation of hematopoietic-depleted FTOC resulted in an upregulation of P-selectin and ICAM-1 adhesion molecules in EC. Third, we demonstrate that EC cellularity, including functional subsets, were substantially reduced in EC^{ΔRANK} transgenic mice in which RANK was selectively deleted in EC. Therefore, these mice showed a reduction in ETP and thymocyte subsets. Collectively, our findings reveal for the first time that RANK expression in EC is crucial for their function and thymic activity. Although LTi and γδ T cells expressed high levels of RANKL, we found using genetically deficient mice that CD4⁺ SP thymocytes are a critical source of RANKL for EC cellularity and functional maturation. These findings are consistent with the higher representation of CD4⁺ SP thymocytes compared to the rare LTi and γδ T cells. Given that ETP also expresses RANKL, it is likely that they also contribute to the functional maturation of EC. In line with this hypothesis, it has been shown that ETP provide proliferative signals to EC via the Kit ligand-c-Kit axis (58).

Whereas RANKL neutralization mimics thymic involution, RANKL administration in aged mice rejuvenates the thymic structure and function. This is characterized by an increased thymic size, accompanied by the loss of keratin-free zones. We found that mTEC^{hi} from aged mice were highly apoptotic compared with those from young mice. Interestingly, RANKL administration reduced the proportion of apoptotic mTEC^{hi}, suggesting that this cytokine acts as a pro-survival signal. In addition to mTEC, TEPC-enriched cells and cTEC also increased upon RANKL administration. In line with this, RANK was substantially expressed in cTEC by both scRNA-seq and flow cytometry. These results are also consistent with the increased cTEC cellularity in mice deficient for OPG, the soluble decoy receptor for RANKL, in which RANK signaling is amplified (56). The increased cTEC cellularity may also be due to an enhanced recruitment of RANKL⁺ ETP and the development of thymocytes beyond the DN1 stage that control cTEC homeostasis (16, 59). Interestingly, RANKL treatment also restored EC cellularity, including TPEC, to similar levels observed in young mice. This is accompanied by an increased expression of functional molecules, comprising lymphotoxin β receptor (LTβR), implicated in TPEC differentiation (31, 60). Although some discrepancies exist between TPEC and high endothelial venules (HEV), the latter controlling T-cell entry into LN, the selective deletion of Ltbr in EC also results in impaired HEV differentiation, associated to a defective expression of ICAM-1 and GlyCAM-1 (61). Interestingly, these two adhesion molecules were upregulated in thymic venous EC upon RANKL treatment. Thus, our data indicate that RANK stimulation in EC is likely upstream of LTβR, a signaling found to upregulate VCAM-1 and ICAM-1 in thymic EC (62). RANKL administration restored the cellularity and functional phenotype of EC and TEC, thereby enhancing (i) progenitor recruitment, which was also promoted by the increased mobilization of BM-derived lymphoid progenitors, and (ii) de novo T-cell production. Since the effects of thymic involution are visible from 3 to 6 months, we provide the proof-of-concept of RANKL treatment efficacy in middle-aged 12-month-old mice (18). Interestingly, RANKL administration was also efficient in regenerating TEC and T-cell production in 18-month-old mice, in whom thymic involution is more severe, indicating that this treatment may be used in older individuals. Overall, our results show that reduced RANKL production alters TEC and EC cellularity and function, thereby contributing to thymic involution, a process that can be reversed by RANKL treatment.

Importantly, we show that RANK expression is conserved in human TEC and EC and that RANKL treatment increased the cellularity of both cell types, including Aire⁺ mTEC, using human TOC. These findings open novel therapeutic opportunities for rejuvenating thymic function in humans. From this perspective, an important parameter was to evaluate the safety of administering RANKL since it controls the development of active osteoclasts (47). In our thymus rejuvenation experimental setup, we did not observe any bone loss. We also show that combining RANKL with bisphosphonates (which prevents bone resorption) constitutes a valuable alternative that does not hamper the benefit of RANKL in thymus rejuvenation (48). Moreover, to avoid any potential side effects, such as osteoporosis, a possible strategy is to deliver RANKL intrathymically in patients under endoscopy guidance, as performed in macaques (63).

Using adoptive transfer of congenic BM cells into unmanipulated aged mice, we show that a single course of RANKL treatment enhanced newly produced CD4⁺ and CD8⁺ T cells in the periphery by approximately 2-fold. To our knowledge, the benefit of thymic rejuvenation in aged mice to improve immune responses remains unclear. We thus hypothesized that RANKL-induced thymic rejuvenation could produce a sufficient amount of T cells to renew the peripheral pool and restore efficient immune responses. In line with this, we found that thymic rejuvenation ameliorates anti-melanoma responses, characterized by a reduced tumor growth and an increased infiltration of CD8⁺ T cells including CTL and Th1 cytotoxic cells. Interestingly, while CD8⁺ T cells from aged controls displayed a strong exhausted PD-1 phenotype, CD8⁺ T cells from RANKL-treated mice showed reduced levels. The efficacy of ICBT in aged patients remains uncertain in many cancers, including melanoma (64, 65). We found that anti-PD-1 treatment did not reduce tumor growth in aged control mice. Moreover, ICBT is only efficient in ˜10-30% of patients and can be associated with significant side effects (66, 67), requiring the identification of other strategies. Remarkably, immune control of tumor growth was more efficient in RANKL-treated mice than in anti-PD-1 treated mice. We observed a slight synergistic effect of RANKL and anti-PD-1 treatment with a final tumor volume that tended to be smaller. Since RANKL-induced thymic rejuvenation boosts T-cell production, we expect a better immunosurveillance against cancer emergence. Additionally, in RANKL-treated aged mice, we found improved T-cell dependent antigen-specific responses upon immunization, opening promising perspectives to ameliorate vaccine responses in aged individuals. Overall, RANKL-induced thymic rejuvenation has the potential to enhance T-cell responses in the context of cancer and vaccination in the elderly.

In summary, our study reveals an unexpected key role of the RANK/RANKL axis in thymic involution. Compared with other molecules that only target TEC or thymocytes (68), the advantage of using RANKL is that it controls multiple facets of thymic rejuvenation. It acts on restoring (i) EC functionality, which controls progenitor recruitment and (ii) the cellularity of TEC, which form specialized niches required for T-cell development. We demonstrate that RANKL administration is a novel and potent treatment to efficiently rejuvenate thymic function and boost T-cell immune responses in the elderly. Overall, this study represents a substantial progress in our understanding of the mechanisms that drive thymic involution and provides a new cytokine-based therapeutic approach in the arsenal of regenerative medicine to rejuvenate the immune system.

### REFERENCES

In Example 2, various references describe the state of the art to which this invention pertains. The disclosures of these references are hereby incorporated by reference into the present disclosure:
1. G. Soto-Heredero, M. M. Gómez De Las Heras, J. I. Escrig-Larena, M. Mittelbrunn, Extremely Differentiated T Cell Subsets Contribute to Tissue Deterioration During Aging. Annu. Rev. Immunol. 2023 41, 181-205 (2023).
2. D. Min, A. Panoskaltsis-Mortari, M. Kuro-o, G. A. Holländer, B. R. Blazar, K. I. Weinberg, Sustained thymopoiesis and improvement in functional immunity induced by exogenous KGF administration in murine models of aging. Blood 109, 2529-2537 (2007).
3. I. K. Chinn, C. C. Blackburn, N. R. Manley, G. D. Sempowski, Changes in primary lymphoid organs with aging. Semin Immunol 24, 309-320 (2012).
4. M. Irla, J. Guenot, G. Sealy, W. Reith, B. A. Imhof, A. Serge, Three-Dimensional Visualization of the Mouse Thymus Organization in Health and Immunodeficiency. The Journal of Immunology 190, 586-596 (2013).
5. J. Charaix, A. Borelli, J. C. Santamaria, L. Chasson, M. Giraud, A. Sergé, M. Irla, Recirculating Foxp3+ regulatory T cells are restimulated in the thymus under Aire control. Cellular and Molecular Life Sciences 79 (2022).
6. D. P. Shanley, D. Aw, N. R. Manley, D. B. Palmer, An evolutionary perspective on the mechanisms of immunosenescence. Trends Immunol 30, 374-381 (2009).
7. J. Baran-Gale, M. D. Morgan, S. Maio, F. Dhalla, I. Calvo-Asensio, M. E. Deadman, A. E. Handel, A. Maynard, S. Chen, F. Green, R. V. Sit, N. F. Neff, S. Darmanis, W. Tan, A. P. May, J. C. Marioni, C. P. Ponting, G. A. Holländer, Ageing compromises mouse thymus function and remodels epithelial cell differentiation. Elife 9, 1-71 (2020).
8. B. F. Haynes, M. L. Markert, G. D. Sempowski, D. D. Patel, L. P. Hale, The role of the thymus in immune reconstitution in aging, bone marrow transplantation and HIV-1 infection. Annu. Rev. Immunol 18, 529-560 (2000).
9. B. V. Kumar, T. J. Connors, D. L. Farber, Human T Cell Development, Localization, and Function throughout Life. Immunity 48, 202-213 (2018).
10. E. Clave, I. L. Araujo, C. Alanio, E. Patin, J. Bergstedt, A. Urrutia, S. Lopez-Lastra, Y. Li, B. Charbit, C. R. Macpherson, M. Hasan, B. L. Melo-Lima, C. Douay, Human thymopoiesis is influenced by a common genetic variant within the TCRA-TCRD locus. Sci Transl Med 10, 2966 (2018).
11. D. Granadier, L. Iovino, S. Kinsella, J. A. Dudakov, Dynamics of thymus function and T cell receptor repertoire breadth in health and disease. Semin Immunopathol 43, 119-134 (2021).
12. M. Mittelbrunn, G. Kroemer, Hallmarks of T cell aging. Nat Immunol 22, 687-698 (2021).
13. J. Srinivasan, J. N. Lancaster, N. Singarapu, L. P. Hale, L. I. R. Ehrlich, E. R. Richie, Age-Related Changes in Thymic Central Tolerance. Front Immunol 12, 1-24 (2021).
14. S. Palmer, L. Albergante, C. C. Blackburn, T. J. Newman, Thymic involution and rising disease incidence with age. Proc Natl Acad Sci USA 115, 1883-1888 (2018).
15. K. A. Kooshesh, B. H. Foy, D. B. Sykes, K. Gustafsson, D. T. Scadden, Health Consequences of Thymus Removal in Adults. N Engl J Med 389, 406-417 (2023).
16. M. Irla, Instructive Cues of Thymic T Cell Selection. Annu Rev Immunol 40, 95-119 (2022).
17. J. E. Cowan, J. Malin, Y. Zhao, M. O. Seedhom, C. Harly, I. Ohigashi, M. Kelly, Y. Takahama, J. W. Yewdell, M. Cam, A. Bhandoola, Myc controls a distinct transcriptional program in fetal thymic epithelial cells that determines thymus growth. Nat Commun 10 (2019).
18. S. Ki, D. Park, H. J. Selden, J. Seita, H. Chung, J. Kim, V. R. Iyer, L. I. R. Ehrlich, Global transcriptional profiling reveals distinct functions of thymic stromal subsets and age-related changes during thymic involution. Cell Rep 9, 402-415 (2014).
19. D. H. D. Gray, N. Seach, T. Ueno, M. K. Milton, A. Liston, A. M. Lew, C. C. Goodnow, R. L. Boyd, Developmental kinetics, turnover, and stimulatory capacity of thymic epithelial cells. Blood 108, 3777-3785 (2006).
20. J. N. Lancaster, D. E. Keatinge-Clay, J. Srinivasan, Y. Li, H. J. Selden, S. Nam, E. R. Richie, L. I. R. Ehrlich, Central tolerance is impaired in the middle-aged thymic environment. Aging Cell (2022).
21. J. L. Bautista, N. T. Cramer, C. N. Miller, J. Chavez, D. I. Berrios, L. E. Byrnes, J. Germino, V. Ntranos, J. B. Sneddon, T. D. Burt, J. M. Gardner, C. J. Ye, M. S. Anderson, A. V. Parent, Single-cell transcriptional profiling of human thymic stroma uncovers novel cellular heterogeneity in the thymic medulla. Nat Commun 12 (2021).
22. Ö. Alpdogan, V. M. Hubbard, O. M. Smith, N. Patel, S. Lu, G. L. Goldberg, D. H. Gray, J. Feinman, A. A. Kochman, J. M. Eng, D. Suh, S. J. Muriglan, R. L. Boyd, M. R. M. Van Den Brink, Keratinocyte growth factor (KGF) is required for postnatal thymic regeneration. Blood 107, 2453-2460 (2006).
23. N. Lopes, H. Vachon, J. Marie, M. Irla, Administration of RANKL boosts thymic regeneration upon bone marrow transplantation. EMBO Mol Med 9, 835-851 (2017).
24. J. A. Dudakov, A. M. Hanash, R. R. Jenq, L. F. Young, A. Ghosh, N. V. Singer, M. L. West, O. M. Smith, A. M. Holland, J. J. Tsai, R. L. Boyd, M. R. M. Van Den Brink, Interleukin-22 drives endogenous thymic regeneration in mice. Science (1979) 335, 91-95 (2012).
25. T. Wertheimer, E. Velardi, J. Tsai, K. Cooper, S. Xiao, C. C. Kloss, K. J. Ottmüller, Z. Mokhtari, C. Brede, P. deRoos, S. Kinsella, B. Palikuqi, M. Ginsberg, L. F. Young, F. Kreines, S. R. Lieberman, A. Lazrak, P. Guo, F. Malard, O. M. Smith, Y. Shono, R. R. Jenq, A. M. Hanash, D. J. Nolan, J. M. Butler, A. Beilhack, N. R. Manley, S. Rafii, J. A. Dudakov, M. R. M. van den Brink, Production of BMP4 by endothelial cells is crucial for endogenous thymic regeneration. Sci Immunol 3 (2018).
26. A. J. Coles, L. Azzopardi, O. Kousin-Ezewu, H. K. Mullay, S. A. J. Thompson, L. Jarvis, J. Davies, S. Howlett, D. Rainbow, J. Babar, T. J. Sadler, J. W. L. Brown, E. Needham, K. May, Z. G. Georgieva, A. E. Handel, S. Maio, M. Deadman, I. Rota, G. Holländer, S. Dawson, D. Jayne, R. Seggewiss-Bernhardt, D. C. Douek, J. D. Isaacs, J. L. Jones, Keratinocyte growth factor impairs human thymic recovery from lymphopenia. JCI Insight 4 (2019).
27. M. Irla, RANK Signaling in the Differentiation and Regeneration of Thymic Epithelial Cells. Front Immunol 11, 1-8 (2021).
28. D. A. Michelson, K. Hase, T. Kaisho, C. Benoist, D. Mathis, Thymic epithelial cells co-opt lineage-defining transcription factors to eliminate autoreactive T cells. Cell 185, 2542-2558.e18 (2022).
29. K. L. Wells, C. N. Miller, A. R. Gschwind, W. Wei, J. D. Phipps, M. S. Anderson, L. M. Steinmetz, Combined transient ablation and single cell rna sequencing reveals the development of medullary thymic epithelial cells. Elife 9, 1-80 (2020).
30. H. Xia, S. Zhong, Y. Zhao, B. Ren, Z. Wang, Y. Shi, Q. Chai, X. Wang, M. Zhu, Thymic Egress Is Regulated by T Cell-Derived LTβR Signal and via Distinct Thymic Portal Endothelial Cells. Front Immunol 12 (2021).
31. Y. Shi, W. Wu, Q. Chai, Q. Li, Y. Hou, H. Xia, B. Ren, H. Xu, X. Guo, C. Jin, M. Lv, Z. Wang, Y. X. Fu, M. Zhu, LTBR controls thymic portal endothelial cells for haematopoietic progenitor cell homing and T-cell regeneration. Nat Commun 7 (2016).
32. M. L. Scimone, I. Aifantis, I. Apostolou, H. Von Bochmer, U. H. Von Andrian, A multistep adhesion cascade for lymphoid progenitor cell homing to the thymus. Proc Natl Acad Sci USA 103, 7006-7011 (2006).
33. A. P. Lepique, S. Palencia, H. Irjala, H. T. Petrie, Characterization of vascular adhesion molecules that may facilitate progenitor homing in the post-natal mouse thymus. Clin Dev Immunol 10, 27-33 (2003).
34. M. Irla, S. Hugues, J. Gill, T. Nitta, Y. Hikosaka, I. R. Williams, F. X. Hubert, H. S. Scott, Y. Takahama, G. A. Holländer, W. Reith, Autoantigen-Specific Interactions with CD4+ Thymocytes Control Mature Medullary Thymic Epithelial Cell Cellularity. Immunity 29, 451-463 (2008).
35. N. Lopes, N. Boucherit, J. C. Santamaria, N. Provin, J. Charaix, P. Ferrier, M. Giraud, M. Irla, Thymocytes trigger self-antigen-controlling pathways in immature medullary thymic epithelial stages. Elife 11, 1-31 (2022).
36. O. Bondareva, B. N. Sheikh, Vascular homeostasis and inflammation in health and disease—lessons from single cell technologies. Int J Mol Sci 21, 1-21 (2020).
37. J. Kalucka, L. P. M. H. de Rooij, J. Goveia, K. Rohlenova, S. J. Dumas, E. Meta, N. V. Conchinha, F. Taverna, L. A. Teuwen, K. Veys, M. García-Caballero, S. Khan, V. Geldhof, L. Sokol, R. Chen, L. Treps, M. Borri, P. de Zeeuw, C. Dubois, T. K. Karakach, K. D. Falkenberg, M. Parys, X. Yin, S. Vinckier, Y. Du, R. A. Fenton, L. Schoonjans, M. Dewerchin, G. Eelen, B. Thienpont, L. Lin, L. Bolund, X. Li, Y. Luo, P. Carmeliet, Single-Cell Transcriptome Atlas of Murine Endothelial Cells. Cell 180, 764-779.e20 (2020).
38. A. Camara, O. G. Cordeiro, F. Alloush, J. Sponsel, M. Chypre, L. Onder, K. Asano, M. Tanaka, H. Yagita, B. Ludewig, V. Flacher, C. G. Mueller, Lymph Node Mesenchymal and Endothelial Stromal Cells Cooperate via the RANK-RANKL Cytokine Axis to Shape the Sinusoidal Macrophage Niche. Immunity 50, 1467-1481.e6 (2019).
39. A. Glasner, S. A. Rose, R. Sharma, H. Gudjonson, T. Chu, J. A. Green, S. Rampersaud, I. K. Valdez, E. S. Andretta, B. S. Dhillon, M. Schizas, S. Dikiy, A. Mendoza, W. Hu, Z.-M. Wang, O. Chaudhary, T. Xu, L. Mazutis, G. Rizzuto, A. Quintanal-Villalonga, P. Manoj, E. de Stanchina, C. M. Rudin, D. Pe'er, A. Y. Rudensky, Conserved transcriptional connectivity of regulatory T cells in the tumor microenvironment informs new combination cancer therapy strategies. Nat Immunol (2023).
40. M. Buono, R. Facchini, S. Matsuoka, S. Thongjuca, D. Waithe, T. C. Luis, A. Giustacchini, P. Besmer, A. J. Mead, S. E. W. Jacobsen, C. Nerlov, A dynamic niche provides Kit ligand in a stage-specific manner to the earliest thymocyte progenitors. Nat Cell Biol 18, 157-167 (2016).
41. D. A. Sultana, J. J. Bell, D. A. Zlotoff, M. E. De Obaldia, A. Bhandoola, Eliciting the T cell fate with Notch. Semin Immunol 22, 254-260 (2010).
42. T. Nagatake, Y. C. Zhao, T. Ito, M. Itoh, K. Kometani, M. Furuse, A. Saika, E. Node, J. Kunisawa, N. Minato, Y. Hamazaki, Selective expression of claudin-5 in thymic endothelial cells regulates the blood-thymus barrier and T-cell export. Int Immunol 33, 171-182 (2021).
43. Y. Hua, G. Vella, F. Rambow, E. Allen, A. Antoranz Martinez, M. Duhamel, A. Takeda, S. Jalkanen, S. Junius, A. Smeets, D. Nittner, S. Dimmeler, T. Hehlgans, A. Liston, F. M. Bosisio, G. Floris, D. Laoui, M. Hollmen, D. Lambrechts, P. Merchiers, J. C. Marine, S. Schlenner, G. Bergers, Cancer immunotherapies transition endothelial cells into HEVs that generate TCF1+ T lymphocyte niches through a feed-forward loop. Cancer Cell 40, 1600-1618.e10 (2022).
44. A. Ivetic, H. L. H. Green, S. J. Hart, L-selectin: A major regulator of leukocyte adhesion, migration and signaling. Front Immunol 10 (2019).
45. S. S. Perry, H. Wang, L. J. Pierce, A. M. Yang, S. Tsai, G. J. Spangrude, L-selectin defines a bone marrow analog to the thymic early T-lineage progenitor. Blood 103, 2990-2996 (2004).
46. O. Kollet, A. Dar, S. Shivtiel, A. Kalinkovich, K. Lapid, Y. Sztainberg, M. Tesio, R. M. Samstein, P. Goichberg, A. Spiegel, A. Elson, T. Lapidot, Osteoclasts degrade endosteal components and promote mobilization of hematopoietic progenitor cells. Nat Med 12, 657-664 (2006).
47. D. L. Lacey, E. Timms, H.-L. Tan, M. J. Kelley, C. R. Dunstan, T. Burgess, R. Elliott, A. Colombero, G. Elliott, S. Scully, H. Hsu, J. Sullivan, N. Hawkins, E. Davy, C. Capparelli, A. Eli, Y.-X. Qian, S. Kaufman, I. Sarosi, V. Shalhoub, G. Senaldi, J. Guo, J. Delaney, W. J. Boyle, Osteoprotegerin Ligand Is a Cytokine that Regulates Osteoclast Differentiation and Activation. Cell 93, 165-176 (1998).
48. Y. Tomimori, K. Mori, M. Koide, Y. Nakamichi, T. Ninomiya, N. Udagawa, H. Yasuda, Evaluation of pharmaceuticals with a novel 50-hour animal model of bone loss. Journal of Bone and Mineral Research 24, 1194-1205 (2009).
49. J. Le Lee, M. A. Linterman, Mechanisms underpinning poor antibody responses to vaccines in ageing. Immunol Lett 241, 1-14 (2022).
50. M. Nagasawa, K. Germar, B. Blom, H. Spits, Human CD5+ Innate Lymphoid Cells Are Functionally Immature and Their Development from CD34+ Progenitor Cells Is Regulated by Id2. Front Immunol 8 (2017).
51. X. Zhu, J. Gui, J. Dohkan, L. Cheng, P. F. Barnes, D. M. Su, Lymphohematopoietic progenitors do not have a synchronized defect with age-related thymic involution. Aging Cell 6, 663-672 (2007).
52. J. Gui, X. Zhu, J. Dohkan, L. Cheng, P. F. Barnes, D. M. Su, The aged thymus shows normal recruitment of lymphohematopoietic progenitors but has defects in thymic epithelial cells. Int Immunol 19, 1201-1211 (2007).
53. M. J. Mccarron, M. Irla, A. Sergé, S. M. H. Soudja, J. C. Marie, Transforming Growth Factor-beta signaling in αβ thymocytes promotes negative selection. Nat Commun 10, 5690 (2019).
54. R. Wang, L. Zhang, X. Zhang, J. Moreno, C. Celluzzi, M. Tondravi, Y. Shi, Regulation of activation-induced receptor activator of NF-κB ligand (RANKL) expression in T cells. Eur J Immunol 32, 1090-1098 (2002).
55. G. J. Fiala, A. Q. Gomes, B. Silva-Santos, From thymus to periphery: Molecular basis of effector γδ-T cell differentiation. Immunol Rev 298, 47-60 (2020).
56. I. S. Khan, M. L. Mouchess, M. L. Zhu, B. Conley, K. J. Fasano, Y. Hou, L. Fong, M. A. Su, M. S. Anderson, Enhancement of an anti-tumor immune response by transient blockade of central T cell tolerance. Journal of Experimental Medicine 211, 761-768 (2014).
57. B. V. Kumar, T. J. Connors, D. L. Farber, Human T Cell Development, Localization, and Function throughout Life. Immunity 48, 202-213 (2018).
58. M. Buono, M. L. Thézénas, A. Ceroni, R. Fischer, C. Nerlov, Bi-directional signaling by membrane-bound KitL induces proliferation and coordinates thymic endothelial cell and thymocyte expansion. Nat Commun 9 (2018).
59. I. Ohigashi, M. Kozai, Y. Takahama, Development and developmental potential of cortical thymic epithelial cells. Immunol Rev 271, 10-22 (2016).
60. A. Borelli, M. Irla, Lymphotoxin: from the physiology to the regeneration of the thymic function. Cell Death Differ 28, 2305-2314 (2021).
61. L. Onder, R. Danuser, E. Scandella, S. Firner, Q. Chai, T. Hehlgans, J. V. Stein, B. Ludewig, Endothelial cell-specific lymphotoxin-β receptor signaling is critical for lymph node and high endothelial venule formation. Journal of Experimental Medicine 210, 465-473 (2013).
62. B. Lucas, K. D. James, E. J. Cosway, S. M. Parnell, A. V. Tumanov, C. F. Ware, W. E. Jenkinson, G. Anderson, Lymphotoxin β Receptor Controls T Cell Progenitor Entry to the Thymus. The Journal of Immunology 197, 2665-2672 (2016).
63. S. C. De Barros, V. S. Zimmermann, N. Taylor, S. Phanie, C. De Barros, R. S. Zimmermann, Hematopoietic Stem Cell Transplantation: Targeting the Thymus. Stem Cells 31, 1245-1251 (2013).
64. R. Elias, J. Morales, Y. Rehman, H. Khurshid, Immune Checkpoint Inhibitors in Older Adults. Curr Oncol Rep 18 (2016).
65. S. K. Wong, C. A. Nebhan, D. B. Johnson, Impact of Patient Age on Clinical Efficacy and Toxicity of Checkpoint Inhibitor Therapy. Front Immunol 12 (2021).
66. G. Morad, B. A. Helmink, P. Sharma, J. A. Wargo, Hallmarks of response, resistance, and toxicity to immune checkpoint blockade. Cell 184, 5309-5337 (2021).
67. X. He, C. Xu, Immune checkpoint signaling and cancer immunotherapy. Cell Res 30, 660-669 (2020).
68. E. Velardi, J. J. Tsai, M. R. M. van den Brink, T cell regeneration after immunological injury. Nat Rev Immunol 21, 277-291 (2021).
69. Y. Shinkai, G. Rathbun, K.-P. Lam, E. M. Oltz, V. Stewart, M. Mendelsohn, J. Charron, M. Datta, F. Young, A. M. Stall, F. W. Alt, RAG-2-Deficient Mice Lack Mature Lymphocytes Owing to Inability to Initiate V(D)J Rearrangement. Cell 68, 855-867 (1992).
70. M. J. Barnden, J. Allison, W. R. Heath, F. R. Carbone, Defective TCR expression in transgenic mice constructed using cDNA-based α- and β-chain genes under the control of heterologous regulatory elements. Immunol Cell Biol 76, 34 40 (1998).
71. S. LeibundGut-Landmann, J. M. Waldburger, C. Reis e Sousa, H. Acha-Orbea, W. Reith, MHC class II expression is differentially regulated in plasmacytoid and conventional dendritic cells. Nat Immunol 5, 899-908 (2004).
72. I. Sörensen, R. H. Adams, A. Gossler, DLL1-mediated Notch activation regulates endothelial identity in mouse fetal arteries. Blood 113, 5680-5688 (2009).
73. D. Rios, M. B. Wood, J. Li, B. Chassaing, A. T. Gewirtz, I. R. Williams, Antigen sampling by intestinal M cells is the principal pathway initiating mucosal IgA production to commensal enteric bacteria. Mucosal Immunol 9, 907-916 (2016).
74. K. A. Knoop, N. Kumar, B. R. Butler, S. K. Sakthivel, R. T. Taylor, T. Nochi, H. Akiba, H. Yagita, H. Kiyono, I. R. Williams, RANKL Is Necessary and Sufficient to Initiate Development of Antigen-Sampling M Cells in the Intestinal Epithelium. The Journal of Immunology 183, 5738-5747 (2009).
75. A. Sergé, A. L. Bailly, M. Aurrand-Lions, B. A. Imhof, M. Irla, For3D: Full organ reconstruction in 3D, an automatized tool for deciphering the complexity of lymphoid organs. J Immunol Methods 424, 32-42 (2015).
76. Y. Hao, S. Hao, E. Andersen-Nissen, W. M. Mauck, S. Zheng, A. Butler, M. J. Lee, A. J. Wilk, C. Darby, M. Zager, P. Hoffman, M. Stoeckius, E. Papalexi, E. P. Mimitou, J. Jain, A. Srivastava, T. Stuart, L. M. Fleming, B. Yeung, A. J. Rogers, J. M. McElrath, C. A. Blish, R. Gottardo, P. Smibert, R. Satija, Integrated analysis of multimodal single-cell data. Cell 184, 3573-3587.e29 (2021).
77. C. S. McGinnis, L. M. Murrow, Z. J. Gartner, DoubletFinder: Doublet Detection in Single-Cell RNA Sequencing Data Using Artificial Nearest Neighbors. Cell Syst 8, 329-337.e4 (2019).
78. D. G. Bunis, J. Andrews, G. K. Fragiadakis, T. D. Burt, M. Sirota, DittoSeq: Universal user-friendly single-cell and bulk RNA sequencing visualization toolkit. Bioinformatics 36, 5535-5536 (2020).
79. J. Alquicira-Hernandez, J. E. Powell, Nebulosa recovers single-cell gene expression signals by kernel density estimation. Bioinformatics 37, 2485-2487 (2021).
80. D. W. Dempster, J. E. Compston, M. K. Drezner, F. H. Glorieux, J. A. Kanis, H. Malluche, P. J. Meunier, S. M. Ott, R. R. Recker, A. M. Parfitt, Standardized nomenclature, symbols, and units for bone histomorphometry: A 2012 update of the report of the ASBMR Histomorphometry Nomenclature Committee. Journal of Bone and Mineral Research 28, 2-17 (2013).

What is claimed is:

1. A method of boosting thymic regeneration in a patient suffering from a thymic injury comprising administering to the patient a therapeutically effective amount of a RANKL polypeptide, wherein the thymic injury results from cytoablative therapy, complications related to HIV/AIDS, aging process, malnutrition, and radiation poisoning due to nuclear disaster.

2. The method of claim 1 wherein the thymic injury is thymic involution due to aging.

3. A method for treating thymic involution in a subject in need thereof comprising administering to the subject a therapeutically effective amount of a RANKL polypeptide.

4. The method of claim 3, wherein the thymic involution is a result of a decline in cellularity and/or function of thymic endothelial cells and/or thymic epithelial cells.

5. The method of claim 3, wherein the step of administering increases cellularity and/or function of thymic endothelial cells and/or thymic epithelial cells.

6. The method of claim 3, wherein the step of administering boosts or enhances long-term recovery of T-cell functions.

7. The method of claim 6, wherein the step of administering boosts or enhances long-term recovery of antitumoral T-cell functions.

8. The method of claim 3 wherein the RANKL polypeptide is a polypeptide comprising an amino acid sequence having at least 80% of identity with SEQ ID NO:1 or SEQ ID NO:2.

9. The method of claim 3 wherein the RANKL polypeptide is fused to an immunoglobulin domain to form an immunoadhesin.

10. The method of claim 9, wherein the immunoglobulin domain is a Fc region.

11. The method of claim 3 wherein the RANKL polypeptide is administered to the patient in combination with bisphosphonate to prevent bone resorption.

12. The method according to claim 3 wherein the patient is a human.

13. A method for prophylactically treating thymic involution in a subject, comprising administering to the subject a therapeutically effective amount of a RANKL polypeptide, wherein the therapeutically effective amount is sufficient to stimulate recovery of long-term T-cell function.

14. The method of claim 13 wherein the RANKL polypeptide is a polypeptide comprising an amino acid sequence having at least 80% of identity with SEQ ID NO:1 or SEQ ID NO:2.

15. The method of claim 13 wherein the RANKL polypeptide is fused to an immunoglobulin domain to form an immunoadhesin.

16. The method of claim 13, wherein the subject has cancer or is susceptible to suffering from cancer.
